(19) United States
(12) Patent Application Publication
(10) Pub. No.: US 2022/0186258 A1
(43) Pub. Date: Jun. 16, 2022

(54) MODIFIED AAV VECTORS THAT DAMPEN THE HUMORAL IMMUNE RESPONSE

(71) Applicant: University of Florida Research Foundation, Incorporated, Gainesville, FL (US)

(72) Inventors: Arun Srivastava, Gainesville, FL (US); Keyun Qing, Newberry, FL (US)

(73) Assignee: University of Florida Research Foundation, Incorporated, Gainesville, FL (US)

(21) Appl. No.: 17/603,302

(22) PCT Filed: Apr. 10, 2020
(86) PCT No.: PCT/US2020/027790

§ 371 Date: Oct. 12, 2021

Related U.S. Application Data
(60) Provisional application No. 62/833,612, filed on Apr. 12, 2019.

(51) Int. Cl. C12N 15/86; A61K 38/21

(52) U.S. Cl. CPC C12N 15/86; C12N 2750/14143; A61K 38/217

(57) ABSTRACT

Provided herein are compositions of recombinant adeno-associated virus (rAAV) particles capable of dampening the humoral immune response against rAAV in the host into which the rAAV particles are introduced. The modified genomes of these rAAV particles comprise heterologous insert nucleic acid and/or inverted terminal repeat (ITR) sequences containing one or more sequences associated with the human leukocyte antigen gene complex DR (HLA-DR) promoter. Also provided herein are methods for transducing host cells with modified rAAV particles to induce a dampened humoral immune responses in order to improve transduction efficiencies. Also provided herein are complexes comprising a modified rAAV particle and a Regulatory Factor X (RFX) transcription factor.

### RELATED APPLICATIONS

This application claims benefit of U.S. Provisional Application No. 62/833,612, filed on Apr. 12, 2019, the entire disclosure of which is incorporated by reference herein.

### BACKGROUND OF THE INVENTION

Adeno-associated virus (AAV) particles are promising as effective gene delivery tools for long term transduction of a desired gene product in a broad range of tissues for numerous diseases and medical conditions. However, humoral immune responses to recombinant AAV (rAAV) particles and gene products present an obstacle to safely and effectively administer AAV particles, including multiple administrations at more than one time point. The first generation AAV vectors of prior rAAV therapies were often neutralized by pre-existing antibodies in the host, especially at high doses.

Many current therapies utilize second generation AAV vectors, in which certain surface-exposed tyrosine residues have been substituted with phenylalanine residues. These substitutions have resulted in 10- to 30-fold increases in transduction efficiencies in mammalian host cells. See Zhong et al., Proc. Natl. Acad. Sci., USA, 105: 7827-7832 (2008). Second-generation AAV2 vectors are less immunogenic than their first generation counterparts, evading capsid-specific T cells. See Martino et al., Blood, 8(121): 2224-2233 (2013). AAV vectors also contain 15 surface-exposed serine and 17 surface-exposed threonine residues that may be phosphorylated, and 10 surface-exposed lysine residues that may be ubiquitinated. Phopshorylation and ubiquitination modifications of these residues, respectively, have led to as high as an 80-fold increase in transduction efficiencies in host cells. See Aslanidi et al., Vaccine, 30: 3908-3917, 2012; Aslanidi et al., PLoS One, 8: e59142, 2013; Li et al., Human Gene Therapy Methods, 26: 211-220, 2015. Most of the surface-exposed tyrosine, serine, threonine, and lysine residues are conserved in AAV serotypes 1 through 10.

Certain second generation AAV vectors have displayed some resistance to neutralization by pooled human immunoglobulins. But rarely does this translate to successful evasion in practice after administration to subjects in the clinic.

Thus, there remains a need to develop modified vectors of multiple serotypes that successfully evade pre-existing antibodies against AAV and reduce the host humoral response after administration in vivo.

### SUMMARY OF THE INVENTION

The present disclosure is based, at least in part, on the finding that pre-existing humoral immunity to AAV can effectively be targeted to reduce immunity to AAV in a subject. It was previously observed that each of the two AAV inverted terminal repeats (ITRs) contain a 20-nucleotide sequence termed the D-sequence that interacts with a cellular protein, termed the D-sequence binding protein (DBP) or double-stranded DBP (dsDBP). See Wang et al., Journal of Virology, 70: 1688-1677 (1996), incorporated herein by reference in its entirety. The inventors found that DBP plays a crucial role in AAV DNA replication and encapsidation.

The inventors recognized that the D-sequence was observed to share a partial sequence homology with the X-box in the regulatory region of the human leukocyte antigen DRA (HLA-DRA) promoter of the human major histocompatibility complex class II (MHC II) genes. The D-sequence was also shown to specifically interact with the regulatory factor binding to the X-box (RFX), the binding of which is a critical step in MHC II gene expression, suggesting that the D-sequence might compete for RFX transcription factor binding, thereby suppressing expression from the HLA-DR promoter. In DNA-mediated transfection experiments, using a reporter gene under the control of the HLA-DRA promoter, D-sequence oligonucleotides were found to inhibit expression of the reporter gene expression in HeLa and 293 cells by approximately 93% and 96%, respectively. No inhibition was observed when non-specific synthetic oligonucleotides were used. D-sequence oligonucleotides had no effect on expression from the cytomegalovirus (CMV) immediate-early gene promoter. The inventors also found that interferon-γ (IFN-7)-mediated activation of MHC II gene expression was also inhibited by D-sequence oligonucleotides as well as following infection with either the wild-type AAV or transduction with recombinant AAV vectors.

The MHC II proteins are actively involved in the T cell-mediated humoral immune response. MHC II proteins present antigens from extracellular pathogens to immune cells for ultimate destruction. In humans, the MHC II proteins are encoded by the HLA gene complex. HLAs corresponding to MHC class II include HLA-DP, HLA-DM, HLA-DOA, HLA-DOB, HLA-DQ, and HLA-DR. The HLA-DR promoter comprises X-box, X2-box and Y-box sequences. The inducible HLA-DR promoter is activated upon binding of an Regulatory Factor X (RFX) transcription factor to the X-box.

The AAV ITR D-sequence shares homology with the X-box sequence of the HLA-DR promoter. The inventors had shown that the DBP binds the X-box sequence, as well as the D-sequence. Based on this binding behavior, the DBP may itself comprise a putative transcription factor. See Kwon et al., AAV-mediated gene transfer: Effect on MHC Class II gene expression. Mol. Ther. 1: S190 (2000), herein incorporated by reference in its entirety.

The inventors recognized that the D-sequence-mediated down-regulation of the MHC II gene expression can be exploited towards the development of novel AAV vectors capable of dampening the host humoral response, which has important implication in the optimal use of these vectors in human gene therapy.

Aspects of this disclosure relate to rAAV genomes that have been engineered to include elements of the HLA-DR promoter for administration to host cells. After these modified genomes are introduced into the host cell, they presumably compete for binding to RFX transcription factors with the host cell's native HLA-DR promoter sequences. This competition reduces the number of RFX factors available to induce expression of HLA-DR.

This in turn reduces the overall expression of HLA-DR proteins, which in turn reduces presentation of AAV antigen to the host's immune cells and dampens the humoral immune response. Accordingly, disclosed herein are rAAV particles comprising an rAAV nucleic acid containing a 5′ inverted terminal repeat (ITR), an insert nucleic acid comprising a transgene, and a 3′ ITR, wherein the insert nucleic acid comprises one or more sequences of, or comprised within, the HLA-DR promoter.

Also disclosed herein are rAAV particles comprising an rAAV nucleic acid comprising a 5′ ITR, an insert nucleic acid that includes a transgene, a 3′ ITR, and one or more X-box sequences of an HLA-DR promoter. Also disclosed herein are rAAV particles comprising an rAAV nucleic acid comprising a 5′ ITR and a 3′ ITR, wherein one or both of the 3′ ITR and the 5′ ITR comprise one or more X2-box and/or Y-box sequences of an HLA-DR promoter.

In some embodiments, the transgene is about 2- to about 5-kb in length. In certain embodiments, the transgene is about 4- to about 5-kb in length. In some embodiments, the nucleic acid vector is a self-complementary (sc) vector.

In some embodiments, the rAAV particle is an AAV2 particle. In other embodiments, the rAAV particle is an AAV3 particle. In other embodiments, the rAAV particle is an AAV6 particle. In some embodiments, the AAV2, AAV3, or AAV6 particle comprises a modified capsid protein comprising substitutions at positions that correspond to a surface-exposed amino acid residues in the wild-type AAV2, AAV3, or AAV6 capsid protein, respectively.

In other embodiments, the rAAV particle is a recombinant AAV1, AAV5, AAV8, AAV9, or AAV10 particle. In particular embodiments, the rAAV particle is a recombinant AAV8 particle. In some embodiments, the recombinant AAV1, AAV5, AAV8, AAV9, or AAV10 particle comprises a modified capsid protein comprising substitutions at positions that correspond to a surface-exposed amino acid residues in the wild-type AAV1, AAV5, AAV8, AAV9, or AAV10 capsid protein, respectively.

In some embodiments, the rAAV compositions disclosed herein are administered to a host cell. In certain embodiments, the host cell is a human cell. In some embodiments, the host cell is a stem cell. In some embodiments, the host cell is a liver, muscle, brain, eye, pancreas, kidney, or hematopoietic stem cell. In some embodiments, the host cell is ex vivo. In some embodiments, the host cell is in situ in a host. In some embodiments, the host cell is in situ in a host and the rAAV is administered to the host to target one or more host cells.

Aspects of the disclosure relate to methods of producing rAAV particles with a dampened immune response in the host cell, and optionally further with increased transduction efficiency. In some embodiments, the rAAV particles are produced by packaging a nucleic acid vector comprising inverted terminal repeat (ITR) sequences of a selected serotype in the presence of a Rep protein of the same serotype. In certain embodiments, these ITR sequences contain one or more X-box, X2-box or Y-box sequences.

Further aspects of this disclosure relate to pharmaceutical compositions comprising the rAAV particles disclosed herein and a pharmaceutically acceptable excipient. In certain embodiments, the pharmaceutical compositions is formulated for injection or topical application to a mammalian eye.

Further aspects of this disclosure relate to methods of transducing a host cell comprising administering an effective amount of the rAAV particles or the pharmaceutical compositions disclosed herein to the cell. In some embodiments, interferon-γ is co-administered with the rAAV particles.

### BRIEF DESCRIPTION OF THE DRAWINGS

The following drawings form part of the present specification and are included to further demonstrate certain aspects of the present disclosure, which can be better understood by reference to one or more of these drawings in combination with the detailed description of specific embodiments presented herein.

FIG. 1 is a schematic representation of the components of the inducible HLA-DR (or MHC II) promoter. Following RFX transcription factor binding to the X-box, transcription factors CREB and NF-Y bind to X2 and Y boxes, respectively, after which CIITA master regulator-mediated expression from the HLA-DR promoter is induced. Binding of the RFX, CREB and NF-Y factors recruits the CIITA master regulator.

FIG. 2 is a schematic representation of the model by which the disclosed rAAV vectors featuring ITR-modified AAV genomes mediate the downregulation of the HLA-DR promoter. After these modified genomes are introduced into the host cell, they presumably compete for binding to RFX transcription factors with the host cell's native HLA-DR promoter sequences. This competition reduces the number of RFX factors available in the nucleus to induce expression of the HLA-DR protein. The RFX may bind to either the 5′ ITR or the 3′ ITR, or both, of the ITR-modified AAV genome when both ITRs comprise an X-box sequence.

FIG. 3 is a schematic representation of the development of an exemplary ITR-modified and HLA-DR-modified rAAV vectors from first and second generation AAV vectors. Here, certain surface-exposed threonine (T), serine (S) and tyrosine (Y) residues of the wild-type (“first generation”) AAV vectors have been substituted with valine (V) and phenylalanine (F) residues, respectively, to form second generation (“NextGen”) vectors with enhanced transduction efficiencies. When an ITR-modified (or “X-box-modified”) AAV genome is combined with a second generation AAV capsid, capsid- and ITR-modified vectors are formed. Likewise, when an HLA-DR-modified AAV genome is combined with a second generation AAV capsid, capsid- and HLA-DR-modified vectors are formed. These vectors exhibit both enhanced transduction efficiencies and a dampened effect on pre-existing AAV immunity. Abbreviations: GOI, gene of interest, HLA-DRp, HLA-DR promoter.

FIGS. 4A-4H show that AAV D-sequence inhibits expression of a firefly luciferase (Fluc) reporter gene operably controlled by the HLA-DR promoter in HeLa and HEK293 cells. Expression of pGL3-HLA-DRIIp-Luc plasmid was analyzed by measurement of fluorescence per microgram of cellular protein. FIG. 4A is a graph that shows that 293 cells were either mock-transfected, or transfected with the vector plasmid or with the HLA-DRAp-Fluc plasmid with or without co-transfection with double-stranded synthetic oligonucleotides specific for AAV D-sequence or the X-box sequence FLuc activity was determined 48 hours post-transfection. FIG. 4B is a graph that shows that synthetic single-stranded oligonucleotides specific for the X-box sequences were used as appropriate controls. p=0.001 FIG. 4C is a graph that shows the expression from the CMV promoter was not affected by either AAV D-sequence, the X-box sequence, or AAV DNA. p=n.s. FIGS. 4D to 4F show the effect of AAV DNA on HLA-DRA promoter-driven FLuc reporter gene expression in 293 and HeLa cells, and effect of interferon-γ on expression from HLA-DRA promoter and its suppression by D-sequence or the X-box sequence in HeLa cells. HEK293 cells (FIG. 4D) and HeLa cells (FIG. 4E) were transfected with either 200 ng of D-sequence or 1 μg of AAV DNA FLuc expression was determined as described in the legend to FIGS. 4A-4C. FIG. 4F shows that HeLa cells were treated with interferon-γ followed by transfection with the vector plasmid or with the HLA-DRAp-FLuc plasmid with or without co-transfection with D-sequence oligonucleotides. IFN-γ-treatment increased the extent of expression from the HLA-DRA promoter. The rest of the steps were the same as described above. p=0.001. FIG. 4G shows that D-sequence-mediated inhibition (such as is shown in FIG. 4A) was not disrupted by the co-administration of interferon-γ to the cell. FIG. 4H shows that luciferase expression operably controlled by a CMV promoter was not inhibited by D-sequence oligonucleotides. Abbeviations: RLU=relative light units; IFN-γ, interferon-γ.

FIG. 5 shows no effect on dose-dependent anti-AAV antibody neutralization as a result of administration of second generation AAV3 vectors in vitro. AAV3QM corresponds to AAV3(Y505+731F+S663V+T492V), and AAV3PM corresponds to AAV3(Y505+731F+S663V+T492V+K533R).

FIG. 6 shows an increase in resistance to dose-dependent anti-AAV antibody titer as a result of administration of second generation AAV6 vectors in vitro. AAV6QM vectors displayed a 10-fold increase in resistance to pooled immunoglobulin (IVIG) neutralization. AAV6TM corresponds to AAV6(Y705+731F+T492V), and AAV6QM corresponds to AAV6(Y705+731F+T492V+S663V).

FIG. 7 shows AAV D-sequence-like sequences that exist in the human genome. Human genomic sequences that share sequence homology with the AAV-D-sequence are shown.

FIG. 8 shows that dsDBP interacts with the X-box of the HLA-DRA promoter. Electrophoretic mobility-shift assays (EMSA) were performed using ³²P end-labeled X-box (CTCCGTTGCTAGGGGAAGGG (SEQ ID NO: 11); lanes 1-5) and the D-sequence (CTCCATCACTAGGGGTTCCT (SEQ ID NO: 12); lanes 6-10) oligonucleotide probes were incubated in the absence (lanes 1, 6) or the presence (lanes 2,7) of WCE. A 200-fold molar excess of unlabeled oligonucleotides specific for the X-box (lanes 3, 8), the D-sequence (lanes 4, 9), or the LT-α upstream regulatory sequences (ATGCATCACTAGGGGTCCAT (SEQ ID NO: 13); lanes 5, 10) were used in competition experiments. The arrow indicates the specific DNA:protein complex with the X-box- or the AAV D-sequence-specific double-stranded oligonucleotides.

FIG. 9 is a schematic structure of a recombinant plasmid containing the firefly luciferase (FLuc) reporter gene under the control of the HLA-DRA promoter. The regulatory structure of the human HLA-DRA promoter is depicted schematically in FIG. 1. Binding of RFX factor to the X-box allows the binding of CREB and NF-Y to X2 and Y boxes, respectively, which allows the binding of a master regulator, CIITA, which regulates HLA-DRA gene expression. An HLA-DRA promoter-driven firefly luciferase reporter plasmid (HLA-DRAp-FLuc), was constructed as described under Materials and Methods.

FIGS. 10A-10B are schematic structures of recombinant AAV vectors containing the wild-type D-sequence (FIG. 10A) and those containing the X-box from the human HLA-DRA promoter (FIG. 10B).

FIG. 11 is a graph showing the evaluation of anti-AAV2 IgG2c antibody titers in C57BL6/J mice following intravenous (IV) or intramuscular (IM) administration of AAV2 vectors containing the wild-type D-sequences or the X-box sequence from the human HLA-DRA promoter. All animal experiments were approved by the Institutional Animal Care and Use Committee (IACUC) and performed according to the guidelines for animal care specified by the Laboratory Animal Resource Center (LARC) at Indiana University School of Medicine, Indianapolis, Ind. Six to 10-week-old C57BL6/J mice were purchased from Jackson Laboratory (Bar Harbor, Me.) and maintained by LARC at Indiana University School of Medicine (Indianapolis, Ind.). Animals were kept in sterile cages until the end of the experiment. C57BL6/J mice (n=4 per group) received intravenous (IV) or intramuscular (IM) injections with 1×10¹⁰ vgs of a self-complementary AAV2-EGFP (scAAV2-EGFP) or scAAV2-X-box-EGFP vectors. Mice were bled three weeks after vector administration and the level of anti-AAV2 IgG2c capsid antibodies were measured by ELISA. A multiple T test comparison between vectors did not show a statistical difference for IV (p=0.806) and IM (p=0.23) injected mice.

FIGS. 12A-12B show the evaluation of transduction efficiency following repeat intravitreal administration of recombinant AAV2 vectors containing the wild-type D-sequences or the human HLA-DRA sequences in C57BL6/J mice.

### DETAILED DESCRIPTION

Aspects of this disclosure relate to rAAV genomes that have been engineered to include elements of the HLA-DR promoter (or “MHC II promoter”) for administration to host cells. The introduction of these modified genomes to host cells cause a reduction in the expression of HLA-DR, which in turn reduces the degree of presentation of AAV antigen by HLA-DR to the host's immune cells. Ultimately, this may dampen the host's overall humoral immune response.

Disclosed herein are recombinant AAV (rAAV) particles comprising an rAAV nucleic acid containing a 5′ inverted terminal repeat (ITR), an insert nucleic acid comprising a transgene, and a 3′ ITR, wherein the rAAV nucleic acid comprises one or more RFX, CREB or NF-Y transcription factor binding sequences (e.g., one or more X-Box, X2-Box, Y-Box, HLA-DR promoter, or other RFX factor binding sequences). The one or more RFX, CREB or NF-Y factor binding sequences can be located in the 5′ ITR, 3′ ITR, and/or the insert nucleic acid. In some embodiments, the disclosed rAAV particles comprise an rAAV nucleic acid containing a 5′ inverted terminal repeat (ITR), an insert nucleic acid comprising a transgene, and a 3′ ITR, wherein the insert nucleic acid comprises an HLA-DR promoter.

Recombinant adeno-associated virus (AAV) vectors have gained significant attention in the past decade as they have shown promise in gene therapy for a number of human diseases, such as in Leber's congenital amaurosis (LCA), lipoprotein lipase deficiency, hemophilia B, aromatic L-amino acid decarboxylase deficiency, choroideremia, Leber hereditary optic neuropathy, hemophilia A, and spinal muscular atrophy (SMA).

Despite these remarkable achievements, a significant obstacle remains in that the presence of pre-existing antibodies to AAV capsids precludes a large population of patients from benefitting from AAV vector-mediated gene therapy since even very low levels of pre-existing antibodies are capable of neutralizing the first generation of AAV vectors. Similarly, at the present time, repeat-dosing with therapeutic AAV vectors is also not possible. Thus, there is a need to develop novel AAV vectors that are capable of either evading the host humoral immune response, or at the very least, dampening it.

Humoral immune response is mediated by antibody production following exposure to antigenic determinants in lymphatic organs, such as B lymphocytes, which are activated and differentiated to form plasma cells, which in turn, synthesize and secrete antibodies specific for an antigen. Major histocompatibility complex II (MHC II) molecules are a class of MHC molecules that are present on professional antigen-presenting cells (APCs), such as dendritic cells, macrophages, and B lymphocytes. MHC II gene expression is regulated by a master regulator, termed, CIITA, expression of which is largely restricted to professional APCs.

The AAV genome contains inverted terminal repeats (ITRs) that are 145 nucleotides in length, the terminal 125 nucleotides (nts) of which are palindromic and form T-shaped hairpin (HP) structures that serve as primers for AAV DNA replication. The ITRs also contain an additional domain, designated the D-sequence, a stretch of 20 nt that is not involved in HP formation. It was previously documented that the D-sequence plays a crucial role in the life cycle of AAV in that (i) the D-sequence is the ‘packaging signal’ for AAV, (ii) the first 10 nts in the D-sequence are indispensable for AAV DNA replication, and (iii) a distinct host cell protein interacts specifically with the double-stranded D-sequence, which has been designated as the double-stranded D-sequence-binding protein [dsDBP].

Since dsDBP forms a specific complex with the AAV D-sequence, it was assumed that DNA sequences with homology to the D-sequence must also exist in the human genome with which this protein might interact. A computer-based homology search revealed that a D-sequence-like sequence exists in the X-box in the regulatory region of the human leukocyte antigen DRA (HLA-DRA) promoter of the human MHC II genes.

As described herein, dsDBP not only interacts with AAV D-sequence, but also with X-box in the HLA-DRA promoter to which the RFX transcription factor is known to bind, suggesting that dsDBP might be a putative RFX transcription factor. AAV D-sequence also strongly and specifically inhibits expression from the HLA-DRA promoter. Interferon-γ (IFN-γ)-mediated activation of MHC II gene expression is also inhibited by D-sequence oligonucleotides and either the wild-type AAV or recombinant AAV vectors. The D-sequence-mediated down-regulation of the MHC II gene expression may be exploited to generate novel AAV vectors to dampen the host humoral response, which have important implications in the optimal use of these vectors in human gene therapy.

As used herein, the term “insert nucleic acid” refers to the nucleotide sequence of the rAAV genome positioned between the 5′ ITR and 3′ ITR sequences. The insert nucleic acid may include one or more transgenes, or heterologous nucleic acids sequence of interest. The transgene may be operably controlled by an HLA-DR promoter. As used herein, the term “HLA-DR-modified” refers to the characteristic of containing an HLA-DR promoter within the insert nucleic acid of the AAV genome, including embodiments wherein a transgene is operably controlled by an HLA-DR promoter.

Also disclosed herein are rAAV particles comprising an rAAV nucleic acid comprising a 5′ ITR, an insert nucleic acid that includes a transgene, a 3′ ITR, and one or more X-box sequences of an HLA-DR promoter. In some embodiments, the one or more X-box sequences of the rAAV particle have been inserted into the 5′ ITR and/or the 3′ ITR. These X-box sequences share homology to the D-sequences of AAV ITRs, a 20-nucleotide sequence that interacts with double-stranded DBP (dsDBP). The D-sequence is defined in the 3′->5′ direction as SEQ ID NO: 10. The nucleotides in bold represent the seven core nucleotides for which the nucleotide sequences of the HLA-DR promoter X-box sequence of SEQ ID NO: 2 below and the D-sequence have complete identity. AGGAACCCCTAGTGATGGAG (SEQ ID NO: 10) [D-sequence]

As used herein, the term “ITR-modified” refers to the characteristic of containing one or more X-box sequences inserted into the ITR(s) or the insert nucleic acid of the AAV genome.

The nucleotide sequences of the human HLA-DR promoter, and its components are as follows (X- and Y-box sequences are italicized, and the X2-box sequence is underlined):

HLA-DR promoter:
(SEQ ID NO: 1)
GGGTACCTCACTAATGTGCTTCAGGTATATCCCTGTCTAGAAGTCAGAT
TGGGGTTAAAGAGTCTGTCCGTGATTGACTAACAGTCTTAAATACTTGA
TTTGTTGTTGTTGTTGTCCTGTTTGTTTAAGAACTTTACTTCTTTATCC
AATGAACGGAGTATCTTGTGTCCTGGACCCTTTGCAAGAACCCTTCCCC
TAGCAACAGATGCGTCATCTCAAAATATTTTTCTGATTGGCCAAAGAGT
AATTGATTTGCATTTTAATGGTCAGACTCTATTACACCCCACATTCTCT
TTTCTTTTATTCTTGTCTGTTCTGCCTCACTCCCGAGCTCTACTGACTC
CCAAAAGAGCGCCCAAGAAGAAACCACCGCGGTGG
HLA-DR promoter, X-box:
(SEQ ID NO: 2)
CCCTTCCCCTAGCAACAGAT
HLA-DR promoter, X2-box:
(SEQ ID NO: 3)
GATGCGTCA
HLA-DR promoter, Y-box:
(SEQ ID NO: 4)
CTGATTGGCC

In some embodiments of the disclosed rAAV nucleic acid vectors, the native D sequence in one or both ITRs is replaced by an X-box sequence (e.g., the X-box sequence of SEQ ID NO: 2). In some embodiments, a single D sequence is replaced with an X-box sequence. In some embodiments, the X box sequences are inserted into the insert nucleic acid region, and the native D sequences are not altered. In some embodiments, the X box sequences are inserted into one or more ITRs, and the native D sequences are not altered. In particular embodiments, the X box sequences are inserted into one or more ITRs proximate to the native D sequences, e.g., positioned 5′ or 3′ of the native D sequence.

The disclosed rAAV nucleic acid sequences may comprise one, two, three, or more than three total X-box sequences. The disclosed rAAV nucleic acid sequences may comprise one, two, three, or more than three X-box sequences in the insert nucleic acid. The disclosed rAAV nucleic acid sequences may comprise one, two, three, or more than three X-box sequences in the ITR regions, i.e., the 3′ ITR and/or the 5′ ITR.

In certain embodiments, the nucleotide sequences of the one or more X-box sequences differ by zero, one or two nucleotides relative to SEQ ID NO: 2. In certain embodiments, the nucleotide sequences of one or more X-box sequences, or variants thereof, differ by three, four or five nucleotides relative to SEQ ID NO: 2. In other embodiments, the nucleotide sequences of one or more X-box sequences, or variants thereof, comprise a stretch of seven nucleotides (“CCCCTAG”), or the “core”, of the D-sequence but otherwise differ by more than five nucleotides relative to the X-box sequence of SEQ ID NO: 2. These differences may comprise nucleotides that have been inserted, deleted, or substituted relative to the sequence of SEQ ID NO: 2. In some embodiments, the disclosed X-box sequences comprise truncations at the 5′ or 3′ end relative to SEQ ID NO: 2. In some embodiments, the sequence of the one or more X-box sequences do not comprise the D-sequence, and are not comprised within the D-sequence, of SEQ ID NO: 10.

Certain embodiments of particles comprising ITR-modified vectors of the present disclosure contain an X-box sequence within each ITR. Certain embodiments of particles comprising ITR-modified vectors contain two X-box sequences within one or both ITRs. Other embodiments contain three X-box sequences within in one or both ITRs.

Also disclosed herein are rAAV particles comprising an rAAV nucleic acid comprising a 5′ ITR, an insert nucleic acid that includes a transgene, a 3′ ITR, and one or more X2-box and/or Y-box sequences of an HLA-DR promoter. In certain embodiments, the one or more X2-box and/or Y-box sequences are in the 5′ ITR and/or the 3′ ITR. In other embodiments, the one or more X2-box and/or Y-box sequences are in the insert nucleic acid. In certain embodiments, the one or more X2-box sequences are different by one to two nucleotides relative to SEQ ID NO: 3. In certain embodiments, the one or more Y-box sequences are different by one to two nucleotides relative to ID NO: 4. In some embodiments, the sequence of the X2-box and/or Y-box sequences are not comprised within the D-sequence of SEQ ID NO: 10.

Certain embodiments of the particles comprising modified vectors of the present disclosure contain a Y-box sequence within each ITR. Certain embodiments of ITR-modified vectors contain two or three Y-box sequences within one or both ITRs.

MHC class II molecules are cell-surface glycoproteins that play a central role in the immune system by presenting peptides to the antigen receptor of CD4+ T cells. Expression of MHC Class II genes is tightly regulated, consistent with their critical role in immunity during development and after birth. Their expression is cell-type specific and mainly restricted to thymic epithelial cells and bone marrow derived antigen-presenting cells (APCs) which include B cells, macrophages and dendritic cells. Levels of class II expression also vary according to the developmental stage of APCs. For example, differentiation of B cells into plasma cells as well as maturation of dendritic cells is characterised by the repression of MHC class II gene expression. Moreover, activated T cells are known to express MHC class II molecules and recent studies provide evidence for a functionally distinct subset of T regulatory cells as defined by HLA-DR expression. Other cell types such as fibroblasts, epithelial and endothelial cells do not express MHC II molecules unless they are exposed to specific stimuli, notably interferon-gamma (IFN-γ). See L. Handunnetthi et al., Regulation of major histocompatibility complex class II gene expression, genetic variation and disease, Genes Immun. 2010; 11(2): 99-112. IFN-γ promotes activation of the HLA-DR promoter.

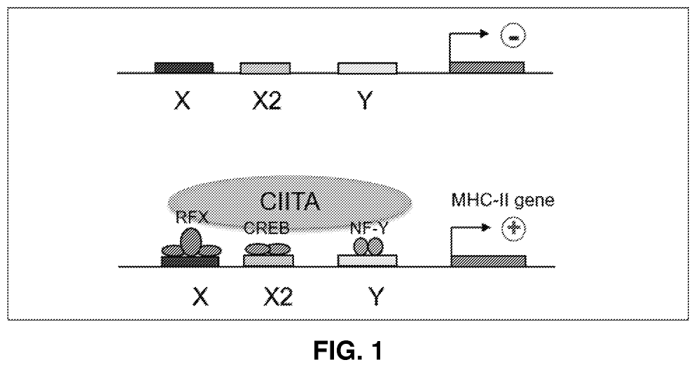

The HLA-DR promoter comprises X-box, X2-box and Y-box sequences (FIG. 1). The X-box sequence binds a trimeric Regulatory Factor X (RFX) transcription factor complex, which is believed to comprise Regulatory Factor X5 (RFX5), RFX-associated protein (RFXAP) and RFX-associated ankyrin-containing protein (RFXANK). Meanwhile, the X2 and Y boxes are bound by cyclic AMP responsive element binding protein (CREB), and nuclear transcription factor Y (NF-Y) respectively. The binding of these promoter sequences by their corresponding transcription factors induces the recruitment of the CIITA protein, which acts as an inducible coactivator to regulate transcription of MHC class II gene expression. See L. Handunnetthi et al., Genes Immun. 2010; 11(2): 99-112.

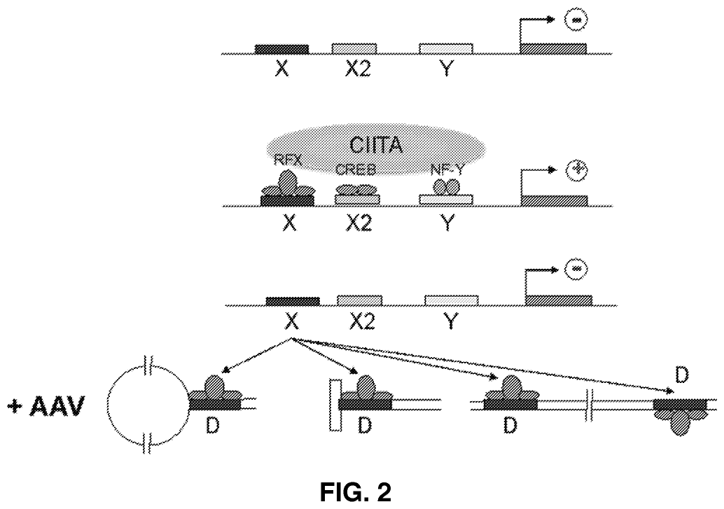

After introduction of capsids with ITR-modified genomes into the host cell, these modified genomes likely compete for binding to RFX transcription factors with the host cell's native HLA-DR promoter sequences, as shown in FIG. 2. This competition reduces the concentration of RFX factors available in the nucleus to bind to the native X-box sequences of the HLA-DR promoter and induce expression of HLA-DR.

Further aspects of this disclosure relate to complexes comprising the rAAV particles disclosed herein and an RFX transcription factor, wherein the RFX is bound to the one or more X-box sequences, X2-box sequences and/or Y-box sequences. In certain embodiments, the RFX is an RFX1 or RFX3 factor.

The degree of binding of a D-sequence or X-box sequence, or an AAV vector comprising one or more D-sequences or X-box sequences, to an RFX factor or variant thereof (or a dsDBP) may be measured by any suitable binding assay known to one of skill in the art (e.g. Electrophoretic Mobility Shift Assay (EMSA).) A triple AAV6 mutant (Y445F+Y731F+F129L) was shown to exhibit 101-fold and 49-fold greater transduction efficiencies than wild-type AAV6 in mouse muscle and lung cells, respectively, further exhibiting a 10-fold increase in resistance to neutralization by pooled human immunoglobulins. See van Lieshout et al., A Novel Triple-Mutant AAV6 Capsid Induces Rapid and Potent Transgene Expression in the Muscle and Respiratory Tract of Mice, Mol. Ther. Methods Clin. Dev. 2018; 9: 323-329, herein incorporated by reference. In addition, a quadruple AAV6 mutant (Y705F+Y731F+T492V+K531R) exhibited capability to evade pre-existing antibodies in human K562 cells. For more information on the quadruple AAV6 mutant, see Ling et al., High-Efficiency Transduction of Primary Human Hematopoietic Stem/Progenitor Cells by AAV6 Vectors: Strategies for Overcoming Donor-Variation and Implications in Genome Editing, Sci. Reports, 6: 35495 (2016) and U.S. Patent Publication No. 2014/0341852, published Nov. 20, 2014, each of which is herein incorporated by reference.

Accordingly, in some embodiments, the rAAV particle is a AAV6 particle. In some embodiments, the AAV6 particle comprises a modified capsid protein comprising a non-tyrosine residue at a position that corresponds to a surface-exposed tyrosine residue in a wild-type AAV6 capsid protein, a non-threonine residue at a position that corresponds to a surface-exposed threonine residue in the wild-type AAV6 capsid protein, a non-lysine residue at a position that corresponds to a surface-exposed lysine residue in the wild-type AAV6 capsid protein, a non-serine residue at a position that corresponds to a surface-exposed serine residue in the wild-type AAV6 capsid protein, or a combination thereof. In some embodiments, the modified capsid protein comprises a non-tyrosine residue and/or a non-threonine residue at one or more of or each of Y705, Y731, and T492 of a wild-type AAV6 capsid protein. In some embodiments, the modified capsid protein comprises a non-tyrosine residue and/or a non-threonine residue and/or a non-serine residue at one or more of or each of Y705, Y731, T492 and S663 of a wild-type AAV6 capsid protein. In some embodiments, the modified capsid protein comprises Y705F, Y731F, T492V and/or K531R substitutions. In some embodiments, the non-tyrosine residue is phenylalanine and the non-threonine residue is valine. In some embodiments, the modified capsid protein comprises Y445F, Y731F and/or F129L substitutions. In some embodiments, the modified capsid protein comprises AAV6QM or AAV6TM.

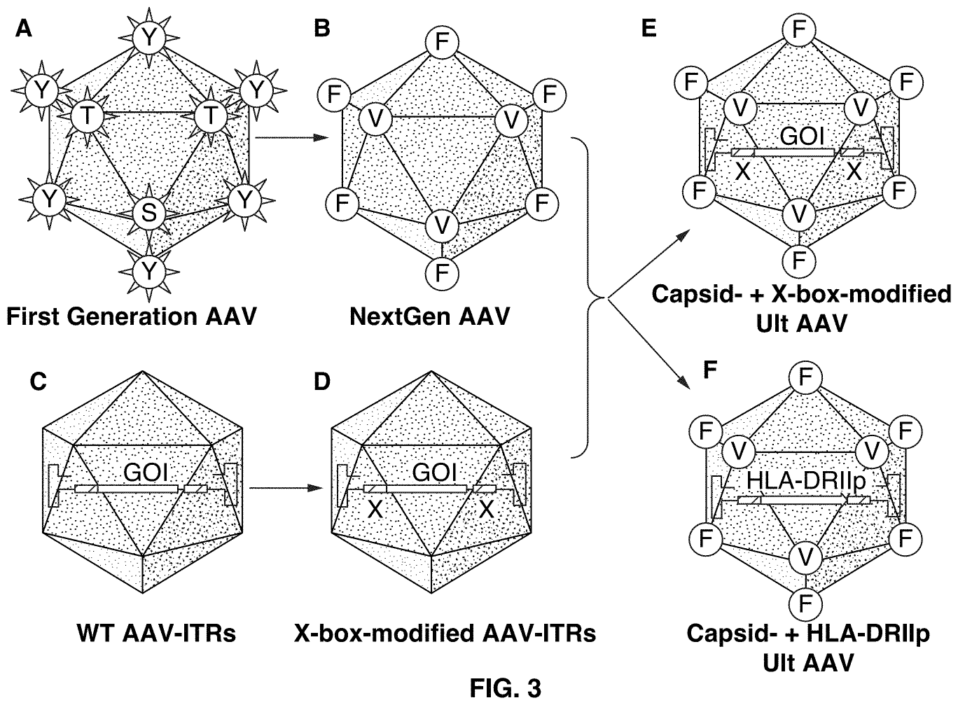

A schematic representation of an exemplary rAAV particle comprising a) a modified capsid protein having substituted tyrosine residues and threonine residues and b) an ITR-modified genome comprising X-box sequence inserts is shown in FIG. 3. These engineered vectors are referred to herein “capsid- and ITR-modified vectors.”

In some embodiments, the rAAV particle is an AAV2 particle. In some embodiments, the AAV2 particle comprises a modified capsid protein comprising a non-tyrosine residue at a position that corresponds to a surface-exposed tyrosine residue in a wild-type AAV2 capsid protein, a non-threonine residue at a position that corresponds to a surface-exposed threonine residue in the wild-type AAV2 capsid protein, a non-lysine residue at a position that corresponds to a surface-exposed lysine residue in the wild-type AAV2 capsid protein, a non-serine residue at a position that corresponds to a surface-exposed serine residue in the wild-type AAV2 capsid protein, or a combination thereof.

In some embodiments, the rAAV particle is an AAV3 particle. In some embodiments, the AAV3 particle comprises a modified capsid protein comprising a non-tyrosine residue at a position that corresponds to a surface-exposed tyrosine residue in a wild-type AAV3 capsid protein, a non-threonine residue at a position that corresponds to a surface-exposed threonine residue in the wild-type AAV3 capsid protein, a non-lysine residue at a position that corresponds to a surface-exposed lysine residue in the wild-type AAV3 capsid protein, a non-serine residue at a position that corresponds to a surface-exposed serine residue in the wild-type AAV3 capsid protein, or a combination thereof. In some embodiments, the modified capsid protein comprises AAV3QM or AAV3PM.

In some embodiments, the rAAV particle is an AAV8 particle. In some embodiments, the modified capsid protein comprises a non-native amino acid substitution at amino acid residue 533 of a wild-type AAV8 capsid, wherein the non-native amino acid substitution is E533K, Y733F, or a combination thereof. The AAV8(Y733F) capsid is described in Doroudchi et al., Amer. Soc. of Gene & Cell Ther. 19(7): 1220-29 (2011), herein incorporated by reference. In certain embodiments, the modified capsid comprises AAV7BP2, a variant of AAV8.

In some embodiments, the transgene encodes a therapeutic protein. In some embodiments, the therapeutic protein is selected from the group consisting of adrenergic agonists, anti-apoptosis factors, apoptosis inhibitors, cytokine receptors, cytokines, cytotoxins, erythropoietic agents, glutamic acid decarboxylases, glycoproteins, growth factors, growth factor receptors, hormones, hormone receptors, interferons, interleukins, interleukin receptors, kinases, kinase inhibitors, nerve growth factors, netrins, neuroactive peptides, neuroactive peptide receptors, neurogenic factors, neurogenic factor receptors, neuropilins, neurotrophic factors, neurotrophins, neurotrophin receptors, N-methyl-D-aspartate antagonists, plexins, proteases, protease inhibitors, protein decarboxylases, protein kinases, protein kinase inhibitors, proteolytic proteins, proteolytic protein inhibitors, semaphorins, semaphorin receptors, serotonin transport proteins, serotonin uptake inhibitors, serotonin receptors, serpins, serpin receptors, and tumor suppressors.

In certain embodiments, the therapeutic protein comprises a Factor IX (FIX) protein, such as a wild-type Factor IX protein or a FIX-Padua mutant. In certain embodiments, the therapeutic protein comprises a Factor VIII (FVIII) protein, such as a wild-type FVIII protein.

Further aspects of this disclosure relate to methods of transducing a host cell comprising administering an effective amount of the rAAV particles or the pharmaceutical compositions disclosed herein to the cell. Any host cell is contemplated for use in a method described herein. In some embodiments, the host cell is a cell in situ in a host, such as a subject as described herein. In some embodiments, the host cell is ex vivo, e.g., in a culture of host cells.

In certain embodiments of the disclosed methods, the rAAV particle is transduced within a mammalian eye cell. The mammalian eye cell may be a human cell. In other embodiments, the host cell is a human cell, a non-human primate cell, a dog cell, a cat cell, a mouse cell, a rat cell, a guinea pig cell, or a hamster cell. In certain embodiments, the mammalian eye cell is selected from the group consisting of an ON retinal bipolar cell, an OFF retinal bipolar cell, a rod bipolar cell, and a cone bipolar cell. In some embodiments, the host cell is a stem cell, such as a hematopoietic stem cell (e.g., a human hematopoietic stem cell). In some embodiments, the host cell is a liver cell, muscle cell, brain cell, eye cell, pancreas cell, or kidney cell.

Pharmaceutical Compositions

As described herein, further provided herein are pharmaceutical compositions that comprise a modified rAAV vector as disclosed herein, and further comprise a pharmaceutical excipient, and may be formulated for administration to host cell ex vivo or in situ in an animal, and particularly a human. Such compositions may further optionally comprise a liposome, a lipid, a lipid complex, a microsphere, a microparticle, a nanosphere, or a nanoparticle, or may be otherwise formulated for administration to the cells, tissues, organs, or body of a subject in need thereof. Such compositions may be formulated for use in a variety of therapies, such as for example, in the amelioration, prevention, and/or treatment of conditions such as peptide deficiency, polypeptide deficiency, peptide overexpression, polypeptide overexpression, including for example, conditions, diseases or disorders as described herein. In particular embodiments, the described compositions may be formulated for use in the amelioration, prevention, and/or treatment of hemophilia A or hemophilia B.

The term “excipient” refers to a diluent, adjuvant, carrier, or vehicle with which the rAAV particle or preparation, or nucleic acid vector is administered. Such pharmaceutical carriers can be sterile liquids, such as water and oils, including those of petroleum oil such as mineral oil, vegetable oil such as peanut oil, soybean oil, and sesame oil, animal oil, or oil of synthetic origin. Saline solutions and aqueous dextrose and glycerol solutions can also be employed as liquid carriers.

In certain embodiments, the present disclosure provides a method of reducing AAV immunity in a subject, wherein the method further comprises administering to the subject a composition comprising the disclosed rAAV particles and a pharmaceutically acceptable excipient, optionally wherein the subject has been previously administered a composition comprising rAAV particles. In particular embodiments, the subject is a human.

In some embodiments, the number of rAAV particles administered to a subject may be on the order ranging from 10⁶ to 10¹⁴ particles/mL or 10³ to 10¹³ particles/mL, or any values therebetween for either range, such as for example, about 10⁶, 10⁷, 10, 10⁹, 10¹⁰, 10¹¹, 10¹², 10¹³, or 10¹⁴ particles/mL. In one embodiment, rAAV particles of higher than 10¹³ particles/mL are be administered. In some embodiments, the number of rAAV particles administered to a subject may be on the order ranging from 10⁶ to 10¹⁴ vector genomes (vgs)/mL or 10³ to 10¹⁵ vgs/mL, or any values there between for either range, such as for example, about 10⁶, 10⁷, 10⁸, 10⁹, 10¹⁰, 10¹¹, 10¹², 10¹³, or 10¹⁴ vgs/mL. In certain embodiments, the methods contain rAAV particle compositions in doses of 3×10³-1×10⁴ vgs/mL. In one embodiment, rAAV particles of higher than 10¹³ vgs/mL are be administered.

The rAAV particles can be administered as a single dose, or divided into two or more administrations as may be required to achieve therapy of the particular disease or disorder being treated (e.g., hemophilia A or hemophilia B). In some embodiments, 0.0001 mL to 10 mLs are delivered to a subject. In some embodiments, interferon-γ is co-administered with the rAAV particles. In some embodiments, interferon-γ is administered after administration of the rAAV particles.

In some embodiments, where a second nucleic acid vector encoding the Rep protein within a second rAAV particle is administered to a subject, the ratio of the first rAAV particle to the second rAAV particle is 1:100, 1:50, 1:40, 1:30, 1:20, 1:10, 1:5, 1:2 or 1:1. In some embodiments, the Rep protein is delivered to a subject such that target cells within the subject receive at least two Rep proteins per cell.

In some embodiments, the disclosure provides formulations of compositions disclosed herein in pharmaceutically acceptable solutions for administration to a cell or an animal, either alone or in combination with one or more other modalities of therapy, and in particular, for therapy of human cells, tissues, and diseases affecting man.

If desired, rAAV particle or preparation, Rep proteins, and nucleic acid vectors may be administered in combination with other agents as well, such as, e.g., proteins or polypeptides or various pharmaceutically-active agents, including one or more systemic or topical administrations of therapeutic polypeptides, biologically active fragments, or variants thereof. In fact, there is virtually no limit to other components that may also be included, given that the additional agents do not cause a significant adverse effect upon contact with the target cells or host tissues. The rAAV particles or preparations, Rep proteins, and nucleic acid vectors may thus be delivered along with various other agents as required in the particular instance. Such compositions may be purified from host cells or other biological sources, or alternatively may be chemically synthesized as described herein. As used herein, the term “vector” can refer to a nucleic acid vector (e.g., a plasmid or recombinant viral genome) or a viral vector (e.g., an rAAV particle comprising a recombinant genome).

Formulation of pharmaceutically-acceptable excipients is well-known to those of skill in the art, as is the development of suitable dosing and treatment regimens for using the particular compositions described herein in a variety of treatment regimens, including e.g., oral, parenteral, intravenous, intranasal, intra-articular, and intramuscular administration and formulation.

Typically, these formulations may contain at least about 0.1% of the therapeutic agent (e.g., rAAV particle or preparation, Rep protein, and/or nucleic acid vector) or more, although the percentage of the active ingredient(s) may, of course, be varied and may conveniently be between about 1 or 2% and about 70% or 80% or more of the weight or volume of the total formulation. Naturally, the amount of therapeutic agent(s) in each therapeutically-useful composition may be prepared is such a way that a suitable dosage will be obtained in any given unit dose of the compound. Factors such as solubility, bioavailability, biological half-life, route of administration, product shelf life, as well as other pharmacological considerations will be contemplated by one skilled in the art of preparing such pharmaceutical formulations, and as such, a variety of dosages and treatment regimens may be desirable.

In certain circumstances it will be desirable to deliver the rAAV particles or preparations, Rep proteins, and/or nucleic acid vectors in suitably formulated pharmaceutical compositions disclosed herein either subcutaneously, intraocularly, intravitreally, parenterally, subcutaneously, intravenously, intracerebro-ventricularly, intramuscularly, intrathecally, orally, intraperitoneally, by oral or nasal inhalation, or by direct injection to one or more cells, tissues, or organs by direct injection.

The pharmaceutical forms of the compositions suitable for injectable use include sterile aqueous solutions or dispersions. In some embodiments, the form is sterile and fluid to the extent that easy syringability exists. In some embodiments, the form is stable under the conditions of manufacture and storage and is preserved against the contaminating action of microorganisms, such as bacteria and fungi. The carrier can be a solvent or dispersion medium containing, for example, water, saline, ethanol, polyol (e.g., glycerol, propylene glycol, and liquid polyethylene glycol, and the like), suitable mixtures thereof, and/or vegetable oils. Proper fluidity may be maintained, for example, by the use of a coating, such as lecithin, by the maintenance of the required particle size in the case of dispersion and by the use of surfactants.

The pharmaceutical compositions of the present disclosure can be administered to the subject being treated by standard routes including, but not limited to, pulmonary, intranasal, oral, inhalation, parenteral such as intravenous, topical, transdermal, intradermal, transmucosal, intraperitoneal, intramuscular, intracapsular, intraorbital, intravitreal, intracardiac, transtracheal, subcutaneous, subcuticular, intraarticular, subcapsular, subarachnoid, intraspinal, epidural and intrasternal injection.

For administration of an injectable aqueous solution, for example, the solution may be suitably buffered, if necessary, and the liquid diluent first rendered isotonic with sufficient saline or glucose. These particular aqueous solutions are especially suitable for intravenous, intramuscular, intravitreal, subcutaneous and intraperitoneal administration. In this connection, a sterile aqueous medium that can be employed will be known to those of skill in the art in light of the present disclosure. For example, one dosage may be dissolved in 1 mL of isotonic NaCl solution and either added to 1000 mL of hypodermoclysis fluid or injected at the proposed site of infusion, (see for example, “Remington's Pharmaceutical Sciences” 15th Edition, pages 1035-1038 and 1570-1580). Some variation in dosage will necessarily occur depending on the condition of the subject being treated. The person responsible for administration will, in any event, determine the appropriate dose for the individual subject. Moreover, for human administration, preparations should meet sterility, pyrogenicity, and the general safety and purity standards as required by, e.g., FDA Office of Biologics standards.

Sterile injectable solutions are prepared by incorporating the rAAV particles or preparations, Rep proteins, and/or nucleic acid vectors, in the required amount in the appropriate solvent with several of the other ingredients enumerated above, as required, followed by filtered sterilization. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, the preferred methods of preparation are vacuum-drying and freeze-drying techniques which yield a powder of the active ingredient plus any additional desired ingredient from a previously sterile-filtered solution thereof.

Ex vivo delivery of cells transduced with rAAV particles or preparations, and/or Rep proteins is also contemplated herein. Ex vivo gene delivery may be used to transplant rAAV-transduced host cells back into the host. A suitable ex vivo protocol may include several steps. For example, a segment of target tissue or an aliquot of target fluid may be harvested from the host and rAAV particles or preparations, and/or Rep proteins may be used to transduce a nucleic acid vector into the host cells in the tissue or fluid. These genetically modified cells may then be transplanted back into the host. Several approaches may be used for the reintroduction of cells into the host, including intravenous injection, intraperitoneal injection, or in situ injection into target tissue. Autologous and allogeneic cell transplantation may be used according to the disclosure.

The amount of rAAV particle or preparation, Rep protein, or nucleic acid vector compositions and time of administration of such compositions will be within the purview of the skilled artisan having benefit of the present teachings. It is likely, however, that the administration of therapeutically-effective amounts of the disclosed compositions may be achieved by a single administration, such as for example, a single injection of sufficient numbers of infectious particles to provide therapeutic benefit to the patient undergoing such treatment. Alternatively, in some circumstances, it may be desirable to provide multiple, or successive administrations of the rAAV particle or preparation, Rep protein, or nucleic acid vector compositions, either over a relatively short, or a relatively prolonged period of time, as may be determined by the medical practitioner overseeing the administration of such compositions.

The composition may include rAAV particles or preparations, Rep proteins, and/or nucleic acid vectors, either alone, or in combination with one or more additional active ingredients, which may be obtained from natural or recombinant sources or chemically synthesized. In some embodiments, rAAV particles or preparations are administered in combination, either in the same composition or administered as part of the same treatment regimen, with a proteasome inhibitor, such as Bortezomib, or hydroxyurea.

To “treat” a disease as the term is used herein, means to reduce the frequency or severity of at least one sign or symptom of a disease or disorder experienced by a subject. The compositions described above are typically administered to a subject in an effective amount, that is, an amount capable of producing a desirable result. The desirable result will depend upon the active agent being administered. For example, an effective amount of a rAAV particle may be an amount of the particle that is capable of transferring a heterologous nucleic acid to a host organ, tissue, or cell.

Toxicity and efficacy of the compositions utilized in methods of the disclosure can be determined by standard pharmaceutical procedures, using either cells in culture or experimental animals to determine the LD₅₀ (the dose lethal to 50% of the population). The dose ratio between toxicity and efficacy the therapeutic index and it can be expressed as the ratio LD₅₀/ED₅₀. Those compositions that exhibit large therapeutic indices are preferred. While those that exhibit toxic side effects may be used, care should be taken to design a delivery system that minimizes the potential damage of such side effects. The dosage of compositions as described herein lies generally within a range that includes an ED₅₀ with little or no toxicity. The dosage may vary within this range depending upon the dosage form employed and the route of administration utilized.

Recombinant AAV (rAAV) Particles and Nucleic Acid Vectors

Aspects of the disclosure relate to recombinant adeno-associated virus (rAAV) particles or preparations of such particles for delivery of one or more nucleic acid vectors comprising a sequence encoding a Rep protein, and/or a protein or polypeptide of interest, into various tissues, organs, and/or cells. In some embodiments, the rAAV particle is delivered to a host cell in the presence of a Rep protein as described herein.

The wild-type AAV genome is a single-stranded deoxyribonucleic acid (ssDNA), either positive- or negative-sensed. The genome comprises two inverted terminal repeats (ITRs), one at each end of the DNA strand, two open reading frames (ORFs): rep and cap between the ITRs, and an insert nucleic acid positioned between the ITRs and optionally comprising a transgene. The rep ORF comprises four overlapping genes encoding Rep proteins required for the AAV life cycle. The cap ORF comprises overlapping genes encoding capsid proteins: VP1, VP2 and VP3, which interact together to form the viral capsid. VP1, VP2 and VP3 are translated from one mRNA transcript, which can be spliced in two different manners: either a longer or shorter intron can be excised resulting in the formation of two isoforms of mRNAs: a ˜2.3 kb- and a ˜2.6 kb-long mRNA isoform. The capsid forms a supramolecular assembly of approximately 60 individual capsid protein subunits into a non-enveloped, T-1 icosahedral lattice capable of protecting the AAV genome. The mature capsid is composed of VP1, VP2, and VP3 (molecular masses of approximately 87, 73, and 62 kDa respectively) in a ratio of about 1:1:10.

Recombinant AAV (rAAV) particles may comprise a nucleic acid vector, which may comprise at a minimum: (a) one or more transgenes comprising a sequence encoding a protein or polypeptide of interest or an RNA of interest (e.g., a siRNA or microRNA), or one or more nucleic acid regions comprising a sequence encoding a Rep protein; and (b) one or more regions comprising inverted terminal repeat (ITR) sequences (e.g., engineered ITR sequences) flanking the one or more nucleic acid regions (e.g., transgenes). In some embodiments, the nucleic acid vector is between 4 kb and 5 kb in size (e.g., 4.2 to 4.7 kb in size). In some embodiments, the nucleic acid vector further comprises a region encoding a Rep protein as described herein. Any nucleic acid vector described herein may be encapsidated by a viral capsid, such as an AAV6 capsid or another serotype (e.g., a serotype that is of the same serotype as the ITR sequences), which may comprises a modified capsid protein as described herein. In some embodiments, the nucleic acid vector is circular. In some embodiments, the nucleic acid vector is single-stranded. In some embodiments, the nucleic acid vector is double-stranded. In some embodiments, a double-stranded nucleic acid vector may be, for example, a self-complimentary vector that contains a region of the nucleic acid vector that is complementary to another region of the nucleic acid vector, initiating the formation of the double-strandedness of the nucleic acid vector.

Accordingly, in some embodiments, an rAAV particle or rAAV preparation containing such particles comprises a viral capsid and a nucleic acid vector as described herein, which is encapsidated by the viral capsid. In some embodiments, the insert nucleic acid of the nucleic acid vector comprises (1) one or more transgenes comprising a sequence encoding a protein or polypeptide of interest, (2) one or more nucleic acid regions comprising a sequence that facilitates expression of the transgene (e.g., a promoter), and (3) one or more nucleic acid regions comprising a sequence that facilitate integration of the transgene (optionally with the one or more nucleic acid regions comprising a sequence that facilitates expression) into the genome of the subject. In certain embodiments, the promoter of the insert nucleic acid comprises a sequence that has at least 90%, at least 95%, or at least 99% identity to an HLA DR-II promoter. In some embodiments, the promoter of the insert nucleic acid comprises a nucleotide sequence that differs from the sequence set forth in SEQ ID NO: 16 by 1, 2, 3, 4, 5, 6, 7, 8, 9, 10, 11, 12, 13, 14, 15, 16, 17, or 18 nucleotides.

By a nucleic acid molecule (e.g., a promoter) comprising a nucleotide sequence having at least, for example, 95% “identity” to a query nucleic acid sequence, it is intended that the nucleotide sequence of the subject nucleic acid molecule is identical to the query sequence except that the subject nucleic acid molecule sequence may include up to five nucleotide alterations per each 100 nucleotides of the query sequence. In other words, to obtain a promoter having a nucleotide sequence at least 95% identical to a reference (query) sequence, up to 5% of the nucleotides in the subject sequence may be inserted, deleted, or substituted with another nucleotide. These alterations of the reference sequence may occur at the 5′ or 3′ ends of the reference sequence or anywhere between those positions, interspersed either individually among nucleotides in the reference sequence or in one or more contiguous groups within the reference sequence.

As a practical matter, whether any particular nucleic acid molecule is at least 80%, 85%, 90%, 95%, 96%, 97%, 98%, or 99% identical to, for instance, the nucleotide sequence of a promoter such as an hOp181opt, can be determined conventionally using known computer programs. A preferred method for determining the best overall match between a query sequence (e.g., a sequence of the present disclosure) and a subject sequence, also referred to as a global sequence alignment, can be determined using the FASTDB or blastn computer program based on the algorithm of Brutlag et al. (Comp. App. Biosci. 6:237-245 (1990)). In a sequence alignment the query and subject sequences are either both nucleotide sequences or both amino acid sequences. The result of said global sequence alignment is expressed as percent identity. Preferred parameters used in a FASTDB amino acid alignment are: Matrix=PAM 0, k-tuple=2, Mismatch Penalty=1, Joining Penalty=20, Randomization Group Length=0, Cutoff Score=1, Window Size=sequence length, Gap Penalty=5, Gap Size Penalty=0.05, Window Size=500 or the length of the subject amino acid sequence, whichever is shorter. Whether a nucleotide is matched/aligned is determined by results of the FASTDB sequence alignment. This percentage is then subtracted from the percent identity, calculated by the above FASTDB program using the specified parameters, to arrive at a final percent identity score. This final percent identity score is what is used for the purposes of the present disclosure. For subject sequences truncated at the 5′ and/or 3′ ends, relative to the query sequence, the percent identity is corrected by calculating the number of nucleotides of the query sequence that are positioned 5′ to or 3′ to the query sequence, which are not matched/aligned with a corresponding subject nucleotide, as a percent of the total bases of the query sequence.

In some embodiments, the nucleic acid vector comprises one or more transgenes comprising a sequence encoding a protein or polypeptide of interest operably linked to a promoter, wherein the one or more transgenes are flanked on each side with an ITR sequence. In some embodiments, the nucleic acid vector further comprises a region encoding a Rep protein as described herein, either contained within the region flanked by ITRs or outside the region or nucleic acid) operably linked to a promoter (e.g. an HLA-DR promoter), wherein the one or more nucleic acid regions. The ITR sequences can be derived from any AAV serotype (e.g., 1, 2, 3, 4, 5, 6, 7, 8, 9, or 10) or can be derived from more than one serotype. In some embodiments, the ITR sequences are derived from AAV2 or AAV6. In some embodiments, the ITR sequences of the first serotype are derived from AAV3, AAV2 or AAV6. In other embodiments, the ITR sequences of the first serotype are derived from AAV1, AAV5, AAV8, AAV9 or AAV10. In some embodiments, the ITR sequences are the same serotype as the capsid (e.g., AAV3 ITR sequences and AAV3 capsid, etc.).

ITR sequences and plasmids containing ITR sequences are known in the art and commercially available (see, e.g., products and services available from Vector Biolabs, Philadelphia, Pa.; Cellbiolabs, San Diego, Calif.; Agilent Technologies, Santa Clara, Ca; and Addgene, Cambridge, Mass.; and Gene delivery to skeletal muscle results in sustained expression and systemic delivery of a therapeutic protein. Kessler P D, et al. Proc Natl Acad Sci USA. 1996; 93(24):14082-7; and Curtis A. Machida, Methods in Molecular Medicine™ Viral Vectors for Gene Therapy Methods and Protocols. 10.1385/1-59259-304-6:201 Humana Press Inc. 2003: Chapter 10, Targeted Integration by Adeno-Associated Virus. Matthew D. Weitzman, Samuel M. Young Jr., Toni Cathomen and Richard Jude Samulski; U.S. Pat. Nos. 5,139,941 and 5,962,313, all of which are incorporated herein by reference).

In some embodiments, the nucleic acid vector comprises a pTR-UF-11 plasmid backbone, which is a plasmid that contains AAV2 ITRs. This plasmid is commercially available from the American Type Culture Collection (ATCC MBA-331).

Exemplary ITR sequences are provided below.

AAV2:
(SEQ ID NO: 5)
TTGGCCACTCCCTCTCTGCGCGCTCGCTCGCTCACTGAGGCCGGGCGAC
CAAAGGTCGCCCGACGCCCGGGCTTTGCCCGGGCGGCCTCAGTGAGCGA
GCGAGCGCGCAGAGAGGGAGTGGCCAACTCCATCACTAGGGGTTCCT
AAV3:
(SEQ ID NO: 6)
TTGGCCACTCCCTCTATGCGCACTCGCTCGCTCGGTGGGGCCTGGCGAC
CAAAGGTCGCCAGACGGACGTGCTTTGCACGTCCGGCCCCACCGAGCGA
GCGAGTGCGCATAGAGGGAGTGGCCAACTCCATCACTAGAGGTATGGC
AAV6:
(SEQ ID NO: 7)
TTGCCCACTCCCTCTATGCGCGCTCGCTCGCTCGGTGGGGCCTGCGGAC
CAAAGGTCCGCAGACGGCAGAGCTCTGCTCTGCCGGCCCCACCGAGCGA
GCGAGCGCGCATAGAGGGAGTGGGCAACTCCATCACTAGGGGTA
AAV5:
(SEQ ID NO: 8)
CTCTCCCCCCTGTCGCGTTCGCTCGCTCGCTGGCTCGTTTGGGGGGGTG
GCAGCTCAAAGAGCTGCCAGACGACGGCCCTCTGGCCGTCGCCCCCCCA
AACGAGCCAGCGAGCGAGCGAACGCGACAGGGGGGAGAGTGCCACACTC
TCAAGCAAGGGGGTTTTGTA

The rAAV particle, nucleic acid vector, and/or Rep protein (in any form contemplated herein) may be delivered in the form of a composition, such as a composition comprising the active ingredient, such as the rAAV particle, nucleic acid vector, and/or Rep protein (in any form contemplated herein), and a therapeutically or pharmaceutically acceptable carrier. The rAAV particles, Rep proteins, or nucleic acid vectors may be prepared in a variety of compositions, and may also be formulated in appropriate pharmaceutical vehicles for administration to human or animal subjects.

Other aspects of the disclosure are directed to methods that involve contacting cells with an rAAV preparation produced by a method described herein. The contacting may be, e.g., ex vivo or in vivo by administering the rAAV preparation to a subject. The rAAV particle or preparation may be delivered in the form of a composition, such as a composition comprising the active ingredient, such as a rAAV particle or preparation described herein, and a therapeutically or pharmaceutically acceptable excipient. The rAAV particles or preparations may be prepared in a variety of compositions, and may also be formulated in appropriate pharmaceutical vehicles for administration to human or animal subjects.

In some embodiments, the nucleic acid vector comprises one or more regions comprising a sequence that facilitates expression of the nucleic acid (e.g., the transgene or the nucleic acid region encoding the Rep protein), e.g., expression control sequences operatively linked to the nucleic acid. Numerous such sequences are known in the art. Non-limiting examples of expression control sequences include promoters, insulators, silencers, response elements, introns, enhancers, initiation sites, termination signals, and poly(A) tails. Any combination of such control sequences is contemplated herein (e.g., a promoter and an enhancer).

To achieve appropriate expression levels of the protein or polypeptide of interest, any of a number of promoters suitable for use in the selected host cell may be employed. The promoter may be, for example, a constitutive promoter, tissue-specific promoter, inducible promoter, or a synthetic promoter.

Inducible promoters and/or regulatory elements may also be contemplated for achieving appropriate expression levels of the protein or polypeptide of interest. Non-limiting examples of suitable inducible promoters include HLA DR-II promoter and those promoters from genes such as cytochrome P450 genes, heat shock protein genes, metallothionein genes, and hormone-inducible genes, such as the estrogen gene promoter.

Tissue-specific promoters and/or regulatory elements are also contemplated herein. Non-limiting examples of such promoters that may be used include species-specific HLA-DR promoters, e.g. a human HLA-DR promoter.

Synthetic promoters are also contemplated herein. A synthetic promoter may comprise, for example, regions of known promoters, regulatory elements, transcription factor binding sites, enhancer elements, repressor elements, and the like.

In some embodiments, a nucleic acid vector described herein may also contain marker or reporter genes, e.g., LacZ or a fluorescent protein such as luciferase.

In some embodiments, the nucleic acid vector comprises one or more transgenes comprising a sequence encoding a protein or polypeptide of interest, such as a therapeutic protein provided in Table 1 or described herein. In certain embodiments, the therapeutic protein comprises a wild-type FIX protein or a FIX-Padua mutant. In certain embodiments, the therapeutic protein comprises a wild-type FVIII protein.

The transgene encoding the protein or polypeptide of interest may be, e.g., a polypeptide or protein of interest provided in Table 1. The sequences of the polypeptide or protein of interest may be obtained, e.g., using the non-limiting National Center for Biotechnology Information (NCBI) Protein IDs or SEQ ID NOs from patent applications provided in Table 1.

TABLE 1
Non-limiting examples of proteins or polypeptides
of interest and associated diseases

| | Non-limiting | Exemplary NCBI |
|---|---|---|
| | Exemplary | Protein IDs or |
| Protein or Polypeptide | diseases | Patent SEQ ID NOs |
| acid alpha-glucosidase | Pompe Disease | NP_000143.2, |
| (GAA) | | NP_001073271.1, |
| | | NP_001073272.1 |
| Methyl CpG binding | Rett syndrome | NP_001104262.1, |
| protein 2 (MECP2) | | NP_004983.1 |
| Aromatic L-amino acid | Parkinson's disease | NP_000781.1, |
| decarboxylase (AADC) | | NP_001076440.1, |
| | | NP_001229815.1, |
| | | NP_001229816.1, |
| | | NP_001229817.1, |
| | | NP_001229818.1, |
| | | NP_001229819.1 |
| Glial cell-derived | Parkinson's disease | NP_000505.1, |
| neurotrophic factor (GDNF) | | NP_001177397.1, |
| | | NP_001177398.1, |
| | | NP_001265027.1, |
| | | NP_954701.1 |
| Cystic fibrosis trans- | Cystic fibrosis | NP_000483.3 |
| membrane conductance | | |
| regulator (CFTR) | | |
| Tumor necrosis factor | Arthritis, Rheumatoid | SEQ ID NO. 1 of |
| receptor fused to an | arthritis | WO2013025079 |
| antibody Fc (TNFR: Fc) | | |
| HIV-1 gag-proΔrt | HIV infection | SEQ ID NOs. 1-5 |
| (tgAAC09) | | of WO2006073496 |
| Sarcoglycan alpha, beta, | Muscular dystrophy | SGCA |
| gamma, delta, epsilon, or | | NP_000014.1, |
| zeta (SGCA, SGCB, SGCG, | | NP_001129169.1 |
| SGCD, SGCE, or SGCZ) | | SGCB |
| | | NP_000223.1 |
| | | SGCG |
| | | NP_000222.1 |
| | | SGCD |
| | | NP_000328.2, |
| | | NP_001121681.1, |
| | | NP_758447.1 |
| | | SGCE |
| | | NP_001092870.1, |
| | | NP_001092871.1, |
| | | NP_003910.1 |
| | | SGCZ |
| | | NP_631906.2 |
| Alpha-1-antitrypsin (AAT) | Hereditary | NP_000286.3, |
| | emphysema | NP_001002235.1, |
| | or Alpha-1- | NP_001002236.1, |
| | antitrypsin | NP_001121172.1, |
| | deficiency | NP_001121173.1, |
| | | NP_001121174.1, |
| | | NP_001121175.1, |
| | | NP_001121176.1, |
| | | NP_001121177.1, |
| | | NP_001121178.1, |
| | | NP_001121179.1 |
| Glutamate decarboxylase | Parkinson's disease | NP_000808.2, |
| 1(GAD1) | | NP_038473.2 |
| Glutamate decarboxylase | Parkinson's disease | NP_000809.1, |
| 2 (GAD2) | | NP_001127838.1 |
| Aspartoacylase (ASPA) | Canavan's disease | NP_000040.1, |
| | | NP_001121557.1 |
| Nerve growth factor (NGF) | Alzheimer's disease | NP_002497.2 |
| Granulocyte-macrophage | Prostate cancer | NP_000749.2 |
| colonystimulating | | |
| factory (GM-CSF) | | |
| Cluster of Differentiation 86 | Malignant melanoma | NP_001193853.1, |
| (CD86 or B7-2) | | NP_001193854.1, |
| | | NP_008820.3, |
| | | NP_787058.4, |
| | | NP_795711.1 |
| Interleukin 12 (IL-12) | Malignant melanoma | NP_000873.2, |
| | | NP_002178.2 |
| neuropeptide Y (NPY) | Parkinson's disease, | NP_000896.1 |
| | epilepsy | |
| ATPase, Ca++ transporting, | Chronic heart failure | NP_001672.1, |
| cardiac muscle, slow twitch | | NP_733765.1 |
| 2 (SERCA2) | | |
| Dystrophin or | Muscular dystrophy | NP_000100.2, |
| Minidystrophin | | NP_003997.1, |
| | | NP_004000.1, |
| | | NP_004001.1, |
| | | NP_004002.2, |
| | | NP_004003.1, |
| | | NP_004004.1, |
| | | NP_004005.1, |
| | | NP_004006.1, |
| | | NP_004007.1, |
| | | NP_004008.1, |
| | | NP_004009.1, |
| | | NP_004010.1, |
| | | NP_004011.2, |
| | | NP_004012.1, |
| | | NP_004013.1, |
| | | NP_004014.1 |
| Ceroid lipofuscinosis | Late infantile neuronal | NP_000382.3 |
| neuronal 2 (CLN2) | ceroidlipofuscinosis | |
| | or Batten's disease | |
| Neurturin (NRTN) | Parkinson's disease | NP_004549.1 |
| N-acetylglucosaminidase, | Sanfilippo syndrome | NP_000254.2 |
| alpha (NAGLU) | (MPSIIIB) | |
| Iduronidase, alpha-1 | MPSI-Hurler | NP_000194.2 |
| (IDUA) | | |
| Iduronate 2-sulfatase (IDS) | MPSII-Hunter | NP_000193.1, |
| | | NP_001160022.1, |
| | | NP_006114.1 |
| Glucuronidase, beta | MPSVII-Sly | NP_000172.2, |
| (GUSB) | | NP_001271219.1 |
| Hexosaminidase A, α | Tay-Sachs | NP_000511.2 |
| polypeptide (HEXA) | | |
| Retinal pigment epithelium- | Leber congenital | NP_000320.1 |
| specific protein 65 kDa | amaurosis | |
| (RPE65) | | |
| Factor IX (FIX) | Hemophilia B | NP_000124.1 |
| FIX-Padua mutant | | |
| Adenine nucleotide | progressive external | NP_001142.2 |
| translocator (ANT-1) | ophthalmoplegia | |
| ApaLI | mitochondrial | YP_007161330.1 |
| | heteroplasmy, | |
| | myoclonic epilepsy | |
| | with ragged red | |
| | fibers (MERRF) or | |
| | mitochondrial | |
| | encephalomyopathy, | |
| | lactic acidosis, and | |
| | stroke-like episodes | |
| | (MELAS) | |
| NADH ubiquinone | Leber hereditary | YP_003024035.1 |
| oxidoreductase subunit 4 | optic | |
| (ND4) | | |
| very long-acyl-CoA | very long-chain acyl- | NP_000009.1, |
| dehydrogenase (VLCAD) | CoA dehydrogenase | NP_001029031.1, |
| | (VLCAD) deficiency | NP_001257376.1, |
| | | NP_001257377.1 |
| short-chain acyl-CoA | short-chain acyl-CoA | NP_000008.1 |
| dehydrogenase (SCAD) | dehydrogenase | |
| | (SCAD) deficiency | |
| medium-chain acyl-CoA | medium-chain acyl- | NP_000007.1, |
| dehydrogenase (MCAD) | CoA dehydrogenase | NP_001120800.1, |
| | (MCAD) deficiency | NP_001272971.1, |
| | | NP_001272972.1, |
| | | NP_001272973.1 |
| Myotubularin 1 (MTM1) | X-linked myotubular | NP_000243.1 |
| | myopathy | |
| Myophosphorylase (PYGM) | McArdle disease | NP_001158188.1, |
| | (glycogen storage | NP_005600.1 |
| | disease type V, | |
| | myophosphorylase | |
| | deficiency) | |
| Lipoprotein lipase (LPL) | LPL deficiency | NP_000228.1 |
| sFLT01 (VEGF/PlGF | Age-related macular | SEQ ID NOs: 2, 8, |
| (placental growth factor) | degeneration | 21, 23, or 25 of |
| binding domain of human | | WO 2009/105669 |
| VEGFR1/Flt-1 | | |
| (hVEGFR1) fused to the Fc | | |
| portion of human IgG(1) | | |
| through a polyglycine linker) | | |
| Glucocerebrosidase (GC) | Gaucher disease | NP_000148.2, |
| | | NP_001005741.1, |
| | | NP_001005742.1, |
| | | NP_001165282.1, |
| | | NP_001165283.1 |
| UDP glucuronosyltransferase | Crigler-Najjar | NP_000454.1 |
| 1 family, polypeptide A1 | syndrome | |
| (UGT1A1) | | |
| Glucose 6-phosphatase | GSD-Ia | NP_000142.2, |
| (G6Pase) | | NP_001257326.1 |
| Ornithine carbamoyl- | OTC deficiency | NP_000522.3 |
| transferase (OTC) | | |
| Cystathionine-beta-synthase | Homocystinuria | NP_000062.1, |
| (CBS) | | NP_001171479.1, |
| | | NP_001171480.1 |
| Factor VIII (FVIII) | Hemophilia A | NP_000123.1, |
| | | NP_063916.1 |
| Hemochromatosis (HFE) | Hemochromatosis | NP_000401.1, |
| | | NP_620572.1, |
| | | NP_620573.1, |
| | | NP_620575.1, |
| | | NP_620576.1, |
| | | NP_620577.1, |
| | | NP_620578.1, |
| | | NP_620579.1, |
| | | NP_620580.1 |
| Low density lipoprotein | Phenylketonuria | NP_000518.1, |
| receptor (LDLR) | (PKU) | NP_001182727.1, |
| | | NP_001182728.1, |
| | | NP_001182729.1, |
| | | NP_001182732.1 |
| Galactosidase, alpha (AGA) | Fabry disease | NP_000160.1 |
| Phenylalanine | Hyper- | NP_000268.1 |
| hydroxylase (PAH) | cholesterolaemia or | |
| | Phenylketonuria | |
| | (PKU) | |
| Propionyl CoA carboxylase, | Propionic acidaemias | NP_000273.2, |
| alpha polypeptide (PCCA) | | NP_001121164.1, |
| | | NP_001171475.1 |

Other exemplary polypeptides or proteins of interest include adrenergic agonists, anti-apoptosis factors, apoptosis inhibitors, cytokine receptors, cytokines, cytotoxins, erythropoietic agents, glutamic acid decarboxylases, glycoproteins, growth factors, growth factor receptors, hormones, hormone receptors, interferons, interleukins, interleukin receptors, kinases, kinase inhibitors, nerve growth factors, netrins, neuroactive peptides, neuroactive peptide receptors, neurogenic factors, neurogenic factor receptors, neuropilins, neurotrophic factors, neurotrophins, neurotrophin receptors, N-methyl-D-aspartate antagonists, plexins, proteases, protease inhibitors, protein decarboxylases, protein kinases, protein kinsase inhibitors, proteolytic proteins, proteolytic protein inhibitors, semaphoring, semaphorin receptors, serotonin transport proteins, serotonin uptake inhibitors, serotonin receptors, serpins, serpin receptors, and tumor suppressors. In some embodiments, the polypeptide or protein of interest is a human protein or polypeptide.

The rAAV particle or particle within an rAAV preparation may be of any AAV serotype, including any derivative or pseudotype (e.g., 1, 2, 3, 4, 5, 6, 7, 8, 9, 10, 2/1, 2/5, 2/8, 2/9, 3/1, 3/5, 3/8, or 3/9). As used herein, the serotype of an rAAV particle refers to the serotype of the capsid proteins of the recombinant virus. In some embodiments, the capsid protein of the rAAV particle is AAV3, AAV2 or AAV6, or a variant thereof. In other embodiments, the capsid protein of the rAAV particle is AAV1, AAV5, AAV8, AAV9, AAV10, or a variant thereof. In some embodiments, the capsid protein of the rAAV particle is not AAV2. In some embodiments, the capsid protein of the rAAV particle is not AAV8.

In some embodiments, the capsid protein is a variant, derivative or pseudotype of a wild-type protein. Non-limiting examples of derivatives and pseudotypes include rAAV2/1, rAAV2/5, rAAV2/8, rAAV2/9, AAV2-AAV3 hybrid, AAVrh.10, AAVhu.14, AAV3a/3b, AAVrh32.33, AAV-HSC15, AAV-HSC17, AAVhu.37, AAVrh.8, CHt-P6, AAV2.5, AAV6.2, AAV2i8, AAV-HSC15/17, AAVM41, AAV9.45, AAV6(Y445F/Y731F), AAV2.5T, AAV-HAE1/2, AAV clone 32/83, AAVShH10, AAV2 (Y->F), AAV8 (Y733F), AAV2.15, AAV2.4, AAVM41, and AAVr3.45. Such AAV serotypes and derivatives/pseudotypes, and methods of producing such derivatives/pseudotypes are known in the art (see, e.g., Asokan A1, Schaffer D V, Samulski R J, The AAV vector toolkit: poised at the clinical crossroads, Mol Ther. 2012; 20(4):699-708). In some embodiments, the rAAV particle is a pseudotyped rAAV particle, which comprises (a) a nucleic acid vector comprising ITRs from one serotype (e.g., AAV2, AAV3) and (b) a capsid comprised of capsid proteins derived from another serotype (e.g., AAV1, AAV2, AAV3, AAV4, AAV5, AAV6, AAV7, AAV8, AAV9, or AAV10). Methods for producing and using pseudotyped rAAV vectors are known in the art (see, e.g., Duan et al., J. Virol., 75:7662-7671, 2001; Halbert et al., J. Virol., 74:1524-1532, 2000; Zolotukhin et al., Methods, 28:158-167, 2002; and Auricchio et al., Hum. Molec. Genet., 10:3075-3081, 2001).

In some embodiments, the rAAV particle comprises a capsid that includes modified capsid proteins (e.g., capsid proteins comprising a modified VP3 region). Methods of producing modified capsid proteins are known in the art (see, e.g., US Patent Publication No. 2013/0310443, incorporated herein by reference in its entirety). In some embodiments, the rAAV particle comprises a modified capsid protein comprising a non-tyrosine residue (e.g., a phenylalanine) at a position that corresponds to a surface-exposed tyrosine residue in a wild-type capsid protein, a non-threonine residue (e.g., a valine) at a position that corresponds to a surface-exposed threonine residue in the wild-type capsid protein, a non-lysine residue (e.g., a glutamic acid) at a position that corresponds to a surface-exposed lysine residue in the wild-type capsid protein, a non-serine residue (e.g., valine) at a position that corresponds to a surface-exposed serine residue in the wild-type capsid protein, or a combination thereof. Exemplary surface-exposed lysine residues include positions that correspond to K258, K321, K459, K490, K507, K527, K572, K532, K544, K549, K556, K649, K655, K665, or K706 of the wild-type AAV2 capsid protein. Exemplary surface-exposed serine residues include positions that correspond to S261, S264, S267, S276, S384, S458, S468, S492, S498, 5578, S658, S662, S668, S707, or S721 of the wild-type AAV2 capsid protein. Exemplary surface-exposed threonine residues include positions that correspond to T251, T329, T330, T454, T455, T503, T550, T592, T581, T597, T491, T671, T659, T660, T701, T713, or T716 of the wild-type AAV2 capsid protein. Exemplary surface-exposed tyrosine residues include positions that correspond to Y252, Y272, Y444, Y500, Y700, Y704, or Y730 of the wild-type AAV2 capsid protein.

In some embodiments, the modified capsid protein comprises a non-tyrosine (e.g., a phenylalanine) residue at one or more of or each of Y705 and Y731 of a wild-type AAV3 capsid protein. In some embodiments, the modified capsid protein comprises a non-serine residue (e.g., valine) and/or a non-threonine residue (e.g., valine) at one or more of or each of S663 and T492 of a wild-type AAV3 capsid protein. In some embodiments, the modified capsid protein comprises a non-serine residue (e.g., valine), a non-threonine residue (e.g., valine), and/or a non-lysine residue (e.g., arginine) at one or more of or each of S663, T492V and K533 of a wild-type AAV3 capsid protein. In some embodiments, the modified capsid protein comprises a non-tyrosine (e.g., a phenylalanine) residue, non-serine residue (e.g., valine), a non-threonine residue (e.g., valine), and/or a non-lysine residue (e.g., arginine) at one or more of or each of Y705, Y731, S663, T492V and K533 of a wild-type AAV3 capsid protein.

In some embodiments, the modified capsid protein comprises a non-tyrosine residue and/or a non-threonine residue at one or more of or each of Y705, Y731, and T492 of a wild-type AAV6 capsid protein (see sequence below with Y705, Y731, and T492 positions underlined, bolded and italicized). In some embodiments, the non-tyrosine residue is phenylalanine and the non-threonine residue is valine:

| (SEQ ID NO: 9) | |
|---|---|
| 1 | MAADGYLPDW LEDNLSEGIR EWWDLKPGAP KPKANQQKQD DGRGLVLPGY |
| 51 | KYLGPFNGLD KGEPVNAADA AALEHDKAYD QQLKAGDNPY LRYNHADAEF |
| 101 | QERLQEDTSF GGNLGRAVFQ AKKRVLEPFG LVEEGAKTAP GKKRPVEQSP |
| 151 | QEPDSSSGIG KTGQQPAKKR LNFGQTGDSE SVPDPQPLGE PPATPAAVGP |
| 201 | TTMASGGGAP MADNNEGADG VGNASGNWHC DSTWLGDRVI TTSTRTWALP |
| 251 | TYNNHLYKQI SSASTGASND NHYFGYSTPW GYFDFNRFHC HFSPRDWQRL |
| 301 | INNNWGFRPK RLNFKLFNIQ VKEVTTNDGV TTIANNLTST VQVFSDSEYQ |
| 351 | LPYVLGSAHQ GCLPPFPADV FMIPQYGYLT LNNGSQAVGR SSFYCLEYFP |
| 401 | SQMLRTGNNF TFSYTFEDVP FHSSYAHSQS LDRLMNPLID QYLYFLNRTQ |
| 451 | NQSGSAQNKD LLFSRGSPAG MSVQPKNWLP GPCYRQQRVS K [symbol] KTDNNNSN |
| 501 | FTWTGASKYN LNGRESIINP GTAMASHKDD KDKFFPMSGV MIFGKESAGA |
| 551 | SNTALDNVMI TDEEEIKATN PVATERFGTV AVNLQSSSTD PATGDVHVMG |
| 601 | ALPGMVWQDR DVYLQGPIWA KIPHTDGHFH PSPLMGGFGL KHPPPQILIK |
| 651 | NTPVPANPPA EFSATKFASF ITQYSTGQVS VEIEWELQKE NSKRWNPEVQ |
| 701 | YTSN [symbol] AKSAN VDFTVDNNGL YTEPRPIGTR [symbol] LTRPL |

In some embodiments, two rAAV particles are contemplated. In some embodiments, the first rAAV particle comprises a nucleic acid vector as described herein (e.g., comprising a one or more transgenes comprising a sequence encoding a protein or polypeptide of interest flanked by ITR sequences), and the second rAAV particle comprises a second nucleic acid vector that contains a region that encodes a Rep protein as described herein. In some embodiments, the second nucleic acid vector further contains ITR sequences flanking the region encoding the Rep protein.

Other aspects of the disclosure relate to a nucleic acid vector, such as a recombinant nucleic acid vector comprising a nucleic acid region encoding a Rep protein as described herein. In some embodiments, the nucleic acid vector further comprises the one or more transgenes comprising a sequence encoding a protein or polypeptide of interest wherein the one or more transgenes are flanked by ITR sequences. In some embodiments, the nucleic acid region encoding the Rep protein is also flanked by the ITR sequences. In some embodiments, the nucleic acid region encoding the Rep protein is outside of the region flanked by the ITR sequences. In some embodiments, the nucleic acid vector is provided in a form suitable for inclusion in a rAAV particle, such as a single-stranded or self-complementary nucleic acid. In some embodiments, the nucleic acid vector is provided in a form suitable for use in a method of producing rAAV particles. For example, in some embodiments, the nucleic acid vector is a plasmid (e.g., comprising an origin of replication (such as an E. coli ORI) and optionally a selectable marker (such as an Ampicillin or Kanamycin selectable marker)).

Production Methods

Methods of producing rAAV particles and nucleic acid vectors are described herein. Other methods are also known in the art and commercially available (see, e.g., Zolotukhin et al. Production and purification of serotype 1, 2, and 5 recombinant adeno-associated viral vectors. Methods 28 (2002) 158-167; and U.S. Patent Publication Nos. US 2007/0015238 and US 2012/0322861, which are incorporated herein by reference; and plasmids and kits available from ATCC and Cell Biolabs, Inc.). For example, a plasmid containing the nucleic acid vector may be combined with one or more helper plasmids, e.g., that contain a rep gene (e.g., encoding Rep78, Rep68, Rep52 and Rep40) and a cap gene (encoding VP1, VP2, and VP3, including a modified VP3 region as described herein), and transfected into a producer cell line such that the rAAV particle can be packaged and subsequently purified.

In some embodiments, the one or more helper plasmids include a first helper plasmid comprising a rep gene and a cap gene and a second helper plasmid comprising a Ela gene, a E1b gene, a E4 gene, a E2a gene, and a VA gene. In some embodiments, the rep gene is a rep gene derived from AAV3, AAV5, or AAV6 and the cap gene is derived from AAV2, AAV3, AAV5, or AAV6 and may include modifications to the gene in order to produce the modified capsid protein described herein. Exemplary AAV Rep protein sequences are provided herein. In some embodiments, the rep gene is a rep gene derived from AAV2 or AAV6 and the cap gene is derived from AAV6 and may include modifications to the gene in order to produce the modified capsid protein described herein. Helper plasmids, and methods of making such plasmids, are known in the art and commercially available (see, e.g., pDM, pDG, pDPlrs, pDP2rs, pDP3rs, pDP4rs, pDP5rs, pDP6rs, pDG(R484E/R585E), and pDP8.ape plasmids from PlasmidFactory, Bielefeld, Germany; other products and services available from Vector Biolabs, Philadelphia, Pa.; Cellbiolabs, San Diego, Calif.; Agilent Technologies, Santa Clara, Ca; and Addgene, Cambridge, Mass.; pxx6; Grimm et al. (1998), Novel Tools for Production and Purification of Recombinant Adenoassociated Virus Vectors, Human Gene Therapy, Vol. 9, 2745-2760; Kern, A. et al. (2003), Identification of a Heparin-Binding Motif on Adeno-Associated Virus Type 2 Capsids, Journal of Virology, Vol. 77, 11072-11081; Grimm et al. (2003), Helper Virus-Free, Optically Controllable, and Two-Plasmid-Based Production of Adeno-associated Virus Vectors of Serotypes 1 to 6, Molecular Therapy, Vol. 7, 839-850; Kronenberg et al. (2005), A Conformational Change in the Adeno-Associated Virus Type 2 Capsid Leads to the Exposure of Hidden VP1 N Termini, Journal of Virology, Vol. 79, 5296-5303; and Moullier, P. and Snyder, R. O. (2008), International efforts for recombinant adeno-associated viral vector reference standards, Molecular Therapy, Vol. 16, 1185-1188).

An exemplary, non-limiting, rAAV particle production method is described next. One or more helper plasmids are produced or obtained, which comprise rep and cap ORFs for the desired AAV serotype and the adenoviral VA, E2A (DBP), and E4 genes under the transcriptional control of their native promoters. In some embodiments, the one or more helper plasmids comprise rep genes for a first serotype (e.g., AAV3, AAV5, and AAV6), cap genes (which may or may not be of the first serotype) and optionally one or more of the adenoviral VA, E2A (DBP), and E4 genes under the transcriptional control of their native promoters. In some embodiments, the one or more helper plasmids comprise cap ORFs (and optionally rep ORFs) for the desired AAV serotype and the adenoviral VA, E2A (DBP), and E4 genes under the transcriptional control of their native promoters. The cap ORF may also comprise one or more modifications to produce a modified capsid protein as described herein. HEK293 cells (available from ATCC®) are transfected via CaPO₄-mediated transfection, lipids or polymeric molecules such as Polyethylenimine (PEI) with the helper plasmid(s) and a plasmid containing a nucleic acid vector described herein. The HEK293 cells are then incubated for at least 60 hours to allow for rAAV particle production. Alternatively, in another example Sf9-based producer stable cell lines are infected with a single recombinant baculovirus containing the nucleic acid vector. As a further alternative, in another example HEK293 or BHK cell lines are infected with a HSV containing the nucleic acid vector and optionally one or more helper HSVs containing rep and cap ORFs as described herein and the adenoviral VA, E2A (DBP), and E4 genes under the transcriptional control of their native promoters. The HEK293, BHK, or Sf9 cells are then incubated for at least 60 hours to allow for rAAV particle production. The rAAV particles can then be purified using any method known the art or described herein, e.g., by iodixanol step gradient, CsCl gradient, chromatography, or polyethylene glycol (PEG) precipitation.

Host Cells

The disclosure also contemplates host cells that comprise at least one of the disclosed rAAV particles or nucleic acid vectors described herein and optionally further comprise a Rep protein (e.g., in the form of a second rAAV particle, an mRNA, or the protein itself). Such host cells include mammalian host cells, with human host cells being preferred, and may be either isolated, in cell or tissue culture. In the case of genetically modified animal models (e.g., a mouse), the transformed host cells may be comprised within the body of a non-human animal itself.

The disclosure also contemplates host cells that comprise at least one of the disclosed rAAV particles or nucleic acid vectors. Such host cells include mammalian host cells, with human host cells being preferred, and may be either isolated, in cell or tissue culture. In the case of genetically modified animal models (e.g., a mouse), the transformed host cells may be comprised within the body of a non-human animal itself. In some embodiments, the host cell is a cancer cell. In some embodiments, the host cell is a liver cell, such as a liver cancer cell.

In certain embodiments, the host cells are HEK293 cells or HeLa cells.

In some embodiments, a host cell as described herein is derived from a subject as described herein. Host cells may be derived using any method known in the art, e.g., by isolating cells from a fluid or tissue of the subject. In some embodiments, the host cells are cultured. Methods for isolating and culturing cells are well known in the art.

Subjects

Aspects of the disclosure relate to methods and preparations for use with a subject, such as human or non-human primate subjects, a host cell in situ in a subject, or a host cell derived from a subject. Non-limiting examples of non-human primate subjects include macaques (e.g., cynomolgus or rhesus macaques), marmosets, tamarins, spider monkeys, owl monkeys, vervet monkeys, squirrel monkeys, baboons, gorillas, chimpanzees, and orangutans. In some embodiments, the subject is a human subject. Other exemplary subjects include domesticated animals such as dogs and cats; livestock such as horses, cattle, pigs, sheep, goats, and chickens; and other animals such as mice, rats, guinea pigs, and hamsters.

In some embodiments, the subject has or is suspected of having a disease that may be treated with gene therapy. In some embodiments, the subject has or is suspected of having a hemoglobinopathy. A hemoglobinopathy is a disease characterized by one or more mutation(s) in the genome that results in abnormal structure of one or more of the globin chains of the hemoglobin molecule. Exemplary hemoglobinopathies include hemolytic anemia, sickle cell disease, and thalassemia. Sickle cell disease is characterized by the presence of abnormal, sickle-chalped hemoglobins, which can result in severe infections, severe pain, stroke, and an increased risk of death. Subjects having sickle cell disease can be identified, e.g., using one or more of a complete blood count, a blood film, hemoglobin electrophoresis, and genetic testing. Thalassemias are a group of autosomal recessive diseases characterized by a reduction in the amount of hemoglobin produced. Symptoms include iron overload, infection, bone deformities, enlarged spleen, and cardiac disease. The subgroups of thalassemias include alpha-thalassemia, beta-thalassemia, and delta thalassemia. Subjects having a thalassemia may be identified, e.g., using one or more of complete blood count, hemoglobin electrophoresis, Fe Binding Capacity, urine urobilin and urobilogen, peripheral blood smear, hematocrit, and genetic testing.

In some embodiments, the subject has or is suspected of having a disease that may be treated with gene therapy. In some embodiments, the subject has or is suspected of having a disease provided in Table 1. In particular embodiments, the subject has or is suspected of having hemophilia A or hemophilia B.

In some embodiments, the subject has or is suspected of having a disease that may be treated with gene therapy. In some embodiments, the subject has or is suspected of having a proliferative disease, such as cancer. The term “cancer” as used herein is defined as disease characterized by the rapid and uncontrolled growth of aberrant cells. Cancer cells can spread locally or through the bloodstream and lymphatic system to other parts of the body. Examples of various cancers include but are not limited to, breast cancer, prostate cancer, ovarian cancer, cervical cancer, skin cancer, pancreatic cancer, colorectal cancer, renal cancer, liver cancer, brain cancer, lymphoma, leukemia, lung cancer and the like. In some embodiments, the cancer is liver cancer. Exemplary liver cancers include, but are not limited to, hepatocellular carcinoma (HCC), cholangiocarcinoma, angiosarcoma, and hepatoblastoma. Subject having cancer can be identified by the skilled medical practitioner, e.g., using methods known in the art including biopsy, cytology, histology, endoscopy, X-ray, Magnetic Resonance Imaging (MRI), ultrasound, CAT scan (computerized axial tomography), genetic testing, and tests for detection of tumor antigens in the blood or urine.

Intravenous Immunoglobulin (IVIG)

In some embodiments, the methods and compositions described herein are accompanied by administration of Intravenous immunoglobulin (IVIG). IVIG can provide the subject with a pool of antibodies to compensate for the loss of HLA-DR expression and/or antigen presentation. In some embodiments, IVIG is pooled, polyvalent, or IgG antibodies extracted from plasma healthy blood donors. Methods for producing IVIG are known in the art (see, e.g., Immune Deficiency Foundation. IDF Patient and Family Handbook: For Primary Immunodeficiency, Disease, 4th Ed. Towson, Md. 2007, and Jolles et al., Clin Exp Immunol. 2005 October; 142(1): 1-11). IVIG is also commercially available (see, e.g., GAMMAGARD LIQUID® and GAMMAGARD S/D® from Baxter Healthcare, GAMMAPLEX® from Bio Products Laboratory, FLEBOGAMMA® from Grifols, OCTAGAM® from Octapharma, PRIVIGEN® from CSL Behring, GAMUNEX® from Talecris Biotherapeutics, GAMMAKED® from Kedrion and BIVIGAM® from Biotest).

Without further elaboration, it is believed that one skilled in the art can, based on the above description, utilize the present disclosure to its fullest extent. The following specific embodiments are, therefore, to be construed as merely illustrative, and not limitative of the remainder of the disclosure in any way whatsoever. All publications cited herein are incorporated by reference for the purposes or subject matter referenced herein.

### EXAMPLES

### Example 1: The AAV D-Sequence Specifically Inhibits Expression from the HLA-DR Promoter

The D-sequence native to the ITRs of AAV genomes has sequence homology with the X-box sequences of the HLA-DR promoter. To verify that AAV D-sequences were capable of downregulating expression of genes operably controlled by the HLA-DR promoter, the HLA-DR promoter-driven expression of a firefly luciferase reporter gene was tested in the presence of double-stranded D-sequence oligonucleotides in human cells in vitro. Plasmid pGL3-HLA-DRIIp-Luc, which has an SV40 Poly(A) tail, was used in this Example. Synthetic single-stranded oligonucleotides ssX-box Primer 1 and ssX-box Primer 2 were used as controls to confirm that transcription factor binding occurs specifically with the double-stranded D-sequences. An HLA-DR X-box oligonucleotide was tested as a positive control. Plasmid and oligonucleotides were transfected into HEK293 and HeLa cells.

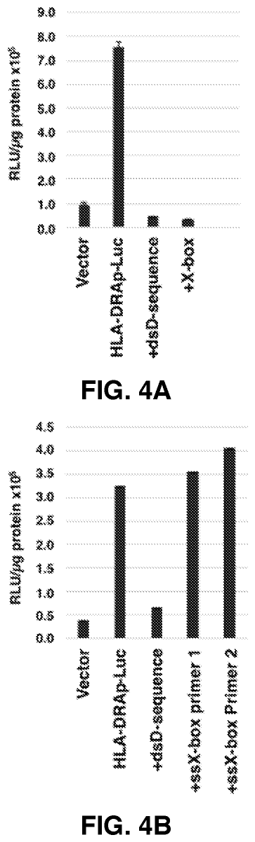

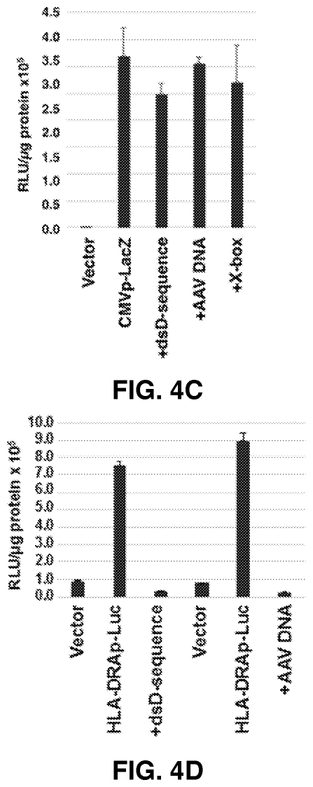

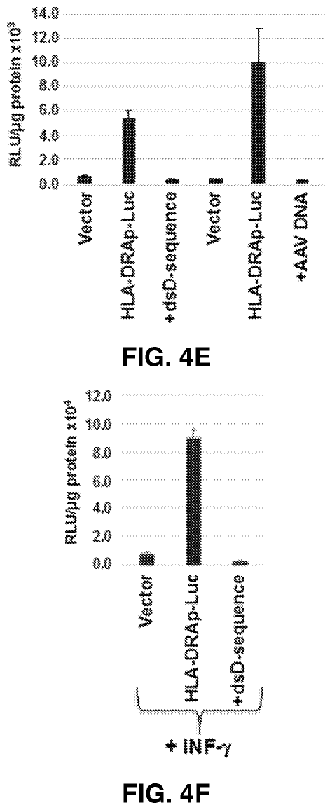

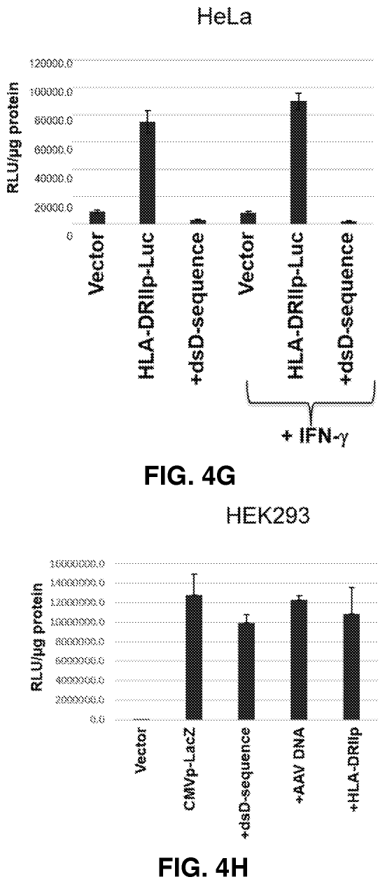

As shown in FIGS. 4A to 4B, luciferase expression in cell lysates was strongly inhibited by the D-sequence oligonucleotides. In contrast, no inhibition was observed by the non-specific synthetic oligonucleotides ssX-box Primer 1 and ssX-box Primer 2. As shown in FIGS. 4D to 4E, luciferase expression was also strongly inhibited by the presence of larger polynucleotides of AAV dsDNA, at levels similar to D-sequences.

The above experiment was repeated in the presence of a LacZ plasmid operably controlled by a CMV promoter. As shown in FIG. 4H, neither the D-sequence oligonucleotide nor the AAV dsDNA polynucleotide induced significant downregulation of LacZ expression by this plasmid.

D-sequence inhibition of HLA-DR-controlled gene expression was strong even in the presence of interferon-γ, known to lead to activation of the HLA-DR promoter and which resulted in enhanced luciferase expression in the absence of any inhibitors. Thus, the experiment shown in FIGS. 4A and 4B was adjusted to include a co-administration of interferon-γ to the cell. Remarkably, as shown in FIG. 4G luciferase expression inhibition by the D-sequence oligonucleotides was unchanged in the presence of interferon-γ.

Accordingly, it was concluded that the D-sequence-mediated inhibition of expression was specific for the HLA-DR promoter. Presumably, the D-sequence competes with X-box sequences to bind to a finite number of RFX transcription factors in vivo.

### Example 2: Evaluation of Capsid-Modified AAV Vectors' Resistance to Antibody Neutralization

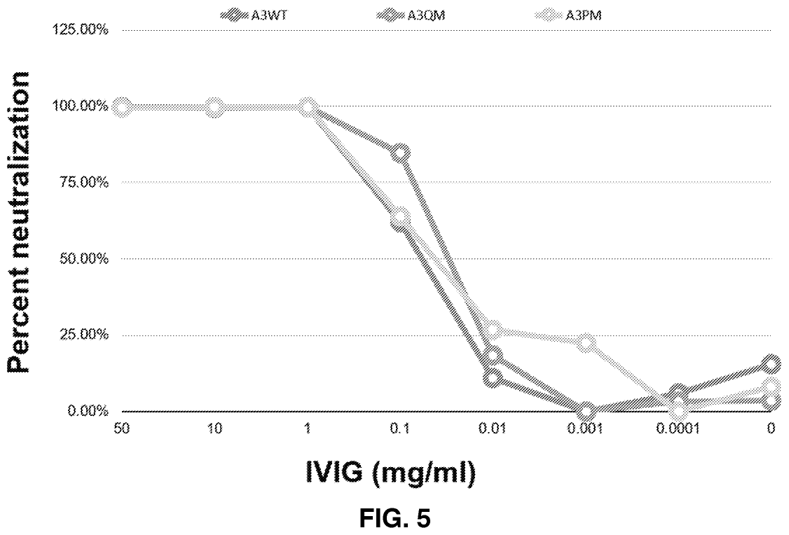

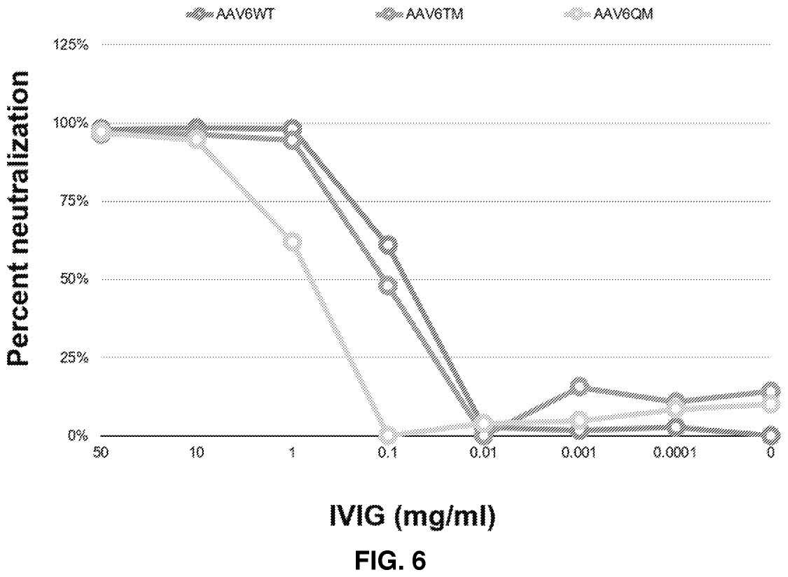

Capsid-modified (second generation) rAAV3 and rAAV6 particles expressing EGFP were pre-treated with pooled immunoglobulins (IVIG) in vitro. EGFP expression was visualized by flow cytometry. As shown in FIG. 5 and FIG. 6, respectively, whereas cells that had been administered modified AAV3 vectors showed no effect on IVIG neutralization, one of the capsid-modified AAV6 vectors, AAV6QM [i.e., AAV6(Y705+731F+T492V+S663V)], displayed a 10-fold increase in resistance to pooled immunoglobulin neutralization relative to wild-type AAV6 (AAV6WT). These results indicate that capsid-modified AAV6 vectors may reduce anti-AAV antibody titer.

### Example 3: Analysis of HLA DR-II-Modified and ITR-Modified AAV Vectors

AAV vectors expressing the EGFP reporter gene under the control of the HLA-DR promoter were generated (GenScript). In addition, AAV vectors expressing the EGFP reporter gene having X-box sequences inserted into the 5′ ITR and 3′ ITR were also generated (GenScript). Studies designed to evaluate the extent of transduction of antigen-presenting cells, such as dendritic cells, B-cells, and macrophages, and efficacy of transduction in murine models in vivo, by these HLA-DR-modified and ITR-modified AAV vectors were completed.

### Example 4: AAV D-Sequence-Mediated Suppression of Expression of a Human Major Histocompatibility Class II Gene

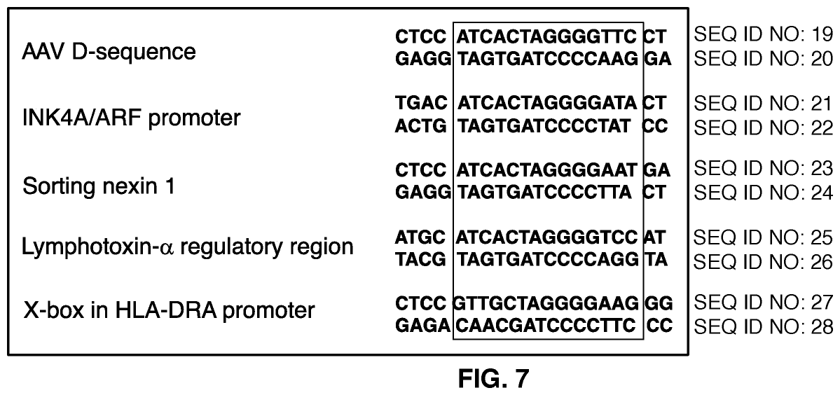

AAV D-sequence shares partial homology with the X-box in the HLA-DRA promoter Since the cellular dsDBP protein formed a specific complex with the AAV D-sequence, it was likely that DNA sequences in the human genome must also exist with which these proteins might interact. Indeed, analysis the database from the National Center for Biotechnology Information (NCBI, http://www.ncbi.nlm.nih.gov) by using blastn program revealed that the D-sequence-like sequences exist in the following cellular genes: (i) INK4A/ARF promoter, (ii) sorting nexin 1 (SNX1), (iii) lymphotoxin-α (LT-α) regulatory region, and (iv) X-box in the HLA-DRA promoter. These sequence alignments are shown in FIG. 7. Several human cellular D-sequence-like sequences were also experimentally obtained by two consecutive rounds of PCR-amplification using commercially available five different human genomic libraries with adapter primers and the AAV D-sequence primer-pairs. PCR amplification products were molecularly cloned in bacterial plasmids and subjected to nucleotide sequencing. Three sets of sequences obtained were homologous with SNX1, LT-α, and HLA-DRA genes. The remarkable similarity of the AAV D-sequence with the X-box sequence in the HLA-DRA promoter, to which a cellular factor, designated RFX, is known to bind, suggested that the dsDBP might share functional similarities with the RFX proteins. Although several D-sequence-like sequences were obtained from human genomic libraries, only the HLA-DRA and LT-α gene sequences were focused on because of this study's focus on the regulation of genes related with immune response to AAV. Thus, all subsequent studies were focused on the X-box in the HLA-DRA gene.

dsDBP Also Interacts with the X-Box in the Human HLA-DRA Promoter

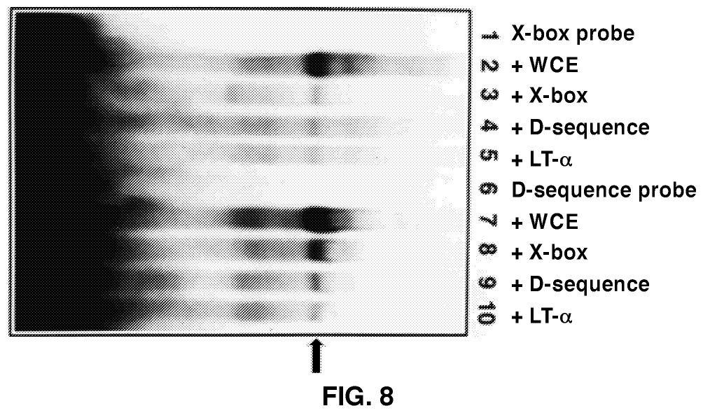

The next step was to determine whether dsDBP also interacted with the X-box. This hypothesis was experimentally tested as follows. Electrophoretic mobility-shift assays (EMSAs) were performed with whole cell extracts (WCE) prepared from 293 cells using radiolabeled X-box- and AAV D-sequence-specific synthetic oligonucleotide probes. These results are shown in FIG. 8. As can be seen, radiolabeled-free X-box (lane 1) and D-sequence (lane 6) formed the same complex with dsDBP (lanes 2 and 7). In cross-competition experiments, where ˜200-fold molar excess of each unlabeled oligonucleotides was used as a competitor, the D-sequence competed for binding with the X-box probe (lane 4), and the X-box sequence competed with the D-sequence probe (lane 8). Competition with the homologous sequences to each of the probes was also observed (lanes 3 and 9). The LT-α sequence competed for binding with the X-box probe (lane 5) and the D-sequence probe (lane 10). Based on these results, the dsDBP is a putative RFX transcription factor. Indeed, RFX1 and RFX3 transcription factors were recently shown to interact with the AAV D-sequence.

### AAV D-Sequence Down-Regulates Transcriptional Activity of the HLA-DRA Promoter Through Inhibition of RFX Binding

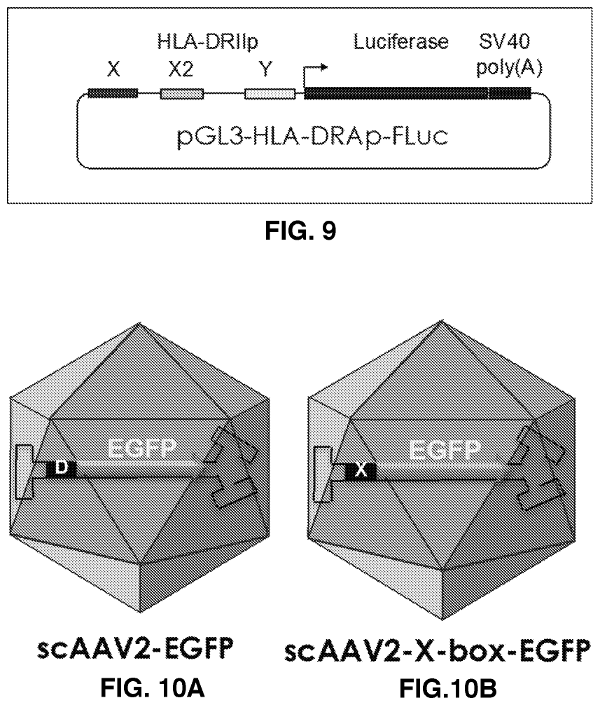

Beyond physical binding of dsDBP to the X-box, the study tested whether the D-sequence competed with the X-box for binding with the RFX transcription factor, which is essential for expression from the HLA DRA promoter (HLA-DRAp). The regulatory structure of the human HLA-DRA promoter is depicted schematically in FIGS. 1 and 2. Briefly, binding of RFX factor to the X-box allows the binding of CREB and NF-Y to X2 and Y boxes, respectively, which allows the binding of a master regulator, CIITA, which regulates HLA-DRA gene expression. An HLA-DRA promoter-driven firefly luciferase reporter plasmid (HLA-DRAp-FLuc), constructed as described above, is depicted schematically in FIG. 9.

HEK 293 cells were either mock-transfected, or transfected with either the vector alone, or with HLA-DRAp-FLuc plasmid with or without various amounts ranging from 10-200 ng of AAV D-sequence- or X-box-specific synthetic double-stranded oligonucleotides. Two additional non-specific synthetic double-stranded oligonucleotides were also used as appropriate controls. FLuc activity was determined 48 hours post-transfection. In preliminary experiments, both D-sequence and X-box oligonucleotides were found to inhibit the reporter gene expression in a dose dependent manner. In subsequent experiments, 200 ng of each oligonucleotides was used. These results are shown in FIGS. 4A to 4F. Both AAV D-sequence and X-box sequences inhibited the reporter gene expression in 293 cells by 93% and ˜96%, respectively (FIG. 4A). No inhibition was detected when two different non-specific synthetic oligonucleotide sequences, or when single-stranded X-box-specific oligonucleotides were used (FIG. 4B). Furthermore, the observed inhibition of expression from the HLA-DRA promoter was specific since the neither D-sequence, nor X-box sequence-specific oligonucleotides had any significant effect on expression of the LacZ reporter gene from the CMV promoter (FIG. 4C). Strong inhibition of expression from the HLA-DRA promoter was also observed with AAV DNA in 293 cells (FIG. 4D) as well as in HeLa cells (FIG. 4E). These results further suggested that AAV D-sequence-mediated down-regulation of the HLA-DR promoter was mediated by recruitment of a putative RFX transcription factor(s) away from the X-box.

### Interferon-γ-Induced MHC II Promoter Activation is Inhibited by AAV D-Sequence

Expression from the endogenous MHC II promoter in HEK 293 or HeLa cells does not normally occur, but in HeLa cells, it can be induced following treatment with IFN-γ. The effect of AAV D-sequence on expression from the HLA-DRA promoter in HeLa cells was examined, with and without pre-treatment with 100 units/ml of IFN-γ. These results are shown in FIG. 4F. As is evident, although IFN-γ-treatment increased the extent of expression from the HLA-DRA promoter, presumably because of activation of the CIITA master regulator, HLA-DRA promoter-mediated expression of the FLuc reporter gene was also significantly inhibited by D-sequence oligonucleotides. These results further corroborated that AAV D-sequence-mediated down-regulation of the HLA-DRA promoter was mediated by recruitment of a putative RFX transcription factor(s) away from the X-box.

Since all the data obtained thus far were derived from DNA transfections, the effect of AAV infection on the MHC II gene expression was evaluated. IFN-γ-treated HeLa cells were either mock-infected, or infected with either the wild-type AAV2 or the recombinant AAV2-LacZ vectors, with and without pre-treatment of cells with tyrphostin 1, known to augment AAV second-strand DNA synthesis, and analyzed for MHC II expression by fluorescence-activated cell sorting (FACS) using mouse anti-human MHC II (DR and DP) monoclonal antibody isotype IgG3 (Chemicon International, Inc., Temecula, Calif.). In preliminary experiments, surface expression of MHC II proteins in interferon-γ-treated HeLa cells was also down-regulated following infection by both wild-type and recombinant AAV (data not shown).

Based on all the available data, a model was proposed, shown in FIG. 2, in which expression from the MHC II promoter does not occur in the absence of RFX transcription factor binding to the X-box. Following RFX transcription factor binding to the X-box, and additional transcription factor binding to X2 and Y boxes, expression from the MHC II promoter ensues. Without being bound to any theory, in the presence of AAV genomes, either as a panhandle structure in a hairpin configuration following viral second-strand DNA synthesis, or in a duplex form following complementary DNA stand annealing, the double-stranded D-sequence effectively competes for the RFX transcription factor binding, thereby rendering the MHC II promoter inactive leading to down-regulation of the MHC II genes. These studies suggested that the D-sequence-mediated down-regulation of the MHC II genes might be exploited toward the development of recombinant AAV vectors capable of dampening the host humoral immune response, which was tested experimentally as follows.

Development of X-Box-Containing AAV Vectors and Evaluation of their Biological Activity:

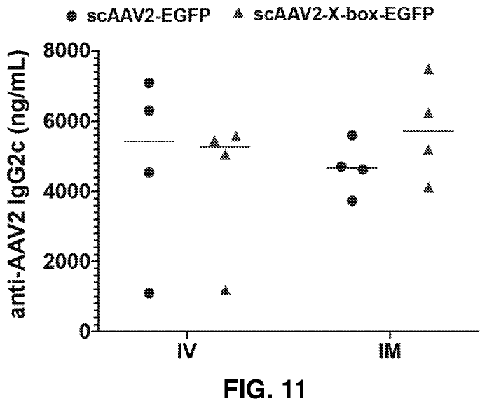

Since the D-sequence in the AAV-ITR, which shares partial sequence homology with the X-box in the HLA-DRA promoter, can functionally compete for the binding of RFX transcription factor, a novel AAV vector in which one D-sequence in the ITR was replaced with an authentic X-box sequence in a self-complementary (scAAV2) vector was generated (FIGS. 10A and 10B). The following two sets of experiments were carried out. In the first set, scAAV2-EGFP or scAAV2-X-box-EGFP vectors were administered, either intravenously or intramuscularly, in C57BL6/J mice (n=4 in each group). Three weeks post-vector administration, the levels of anti-AAV antibody production were determined. These data are shown in FIG. 11. No significant differences were observed in the levels of anti-AAV2 IgG2c antibodies under either experimental condition. The scAAV2-EGFP vector has been described previously and the scAAV2-X-box-EGFP vector was generated as described below.

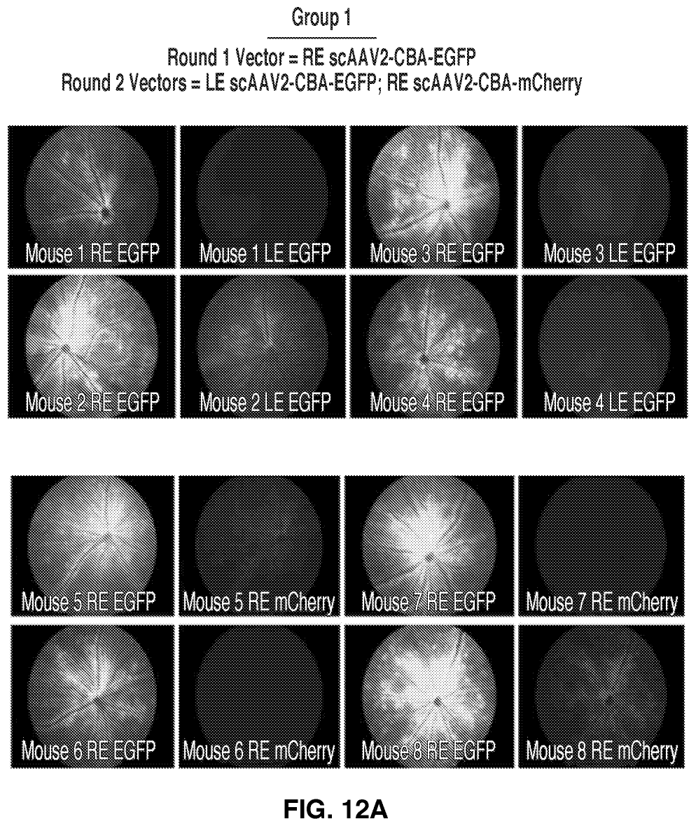

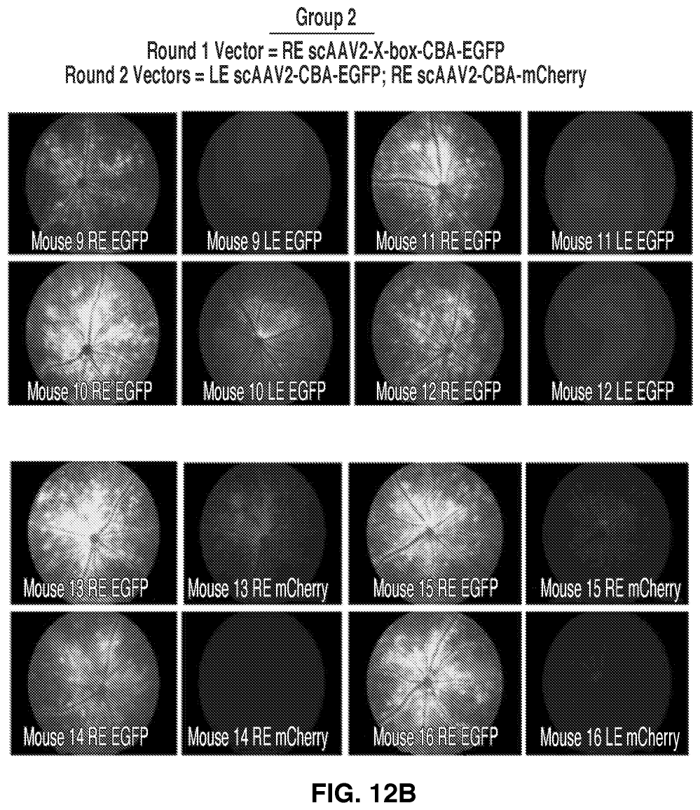

In the second set of experiments, attempts were made to determine whether repeat intravitreal administration of AAVs would be facilitated by initial injection with an X-box containing vector. scAAV2-X-box-EGFP vectors in one eye, transgene expression from a second scAAV-mCherry vectors in the contra-lateral eye could be observed. To this end, two groups of C57BL6/J mice (n=8 in each group) received intravitreal injections in their right eye with scAAV2-EGFP or scAAV2-X-box-EGFP vectors. Four-weeks later, fundus images were acquired, to document the extent of GFP expression. Next, both groups of mice were further sub-divided into two groups (n=4 each). In the first group of 4 mice, scAAV2-EGFP vector was intravitreally injected into the contralateral (left) eye. In the second group of 4 mice, scAAV2-mCherry vector was intravitreally injected in the same (right) eye. This was done to determine whether initial administration of scAAV2-X-box-EGFP would facilitate transgene expression by a second intravitreally-delivered AAV in either the contralateral or same eye, respectively. Four weeks later (and 8 weeks post initial injections), fundus images were captured as described above. These images are shown in FIGS. 12A to 12B. As can be seen, the majority of eyes that received the second intravitreal injection failed to express either EGFP (contralateral eye), or mCherry (same eye), suggesting that either the scAAV2-X-box-EGFP vectors were unable to dampen the humoral immune response, or the subsequent scAAV2-mCherry and scAAV2-GFP vectors failed to overcome antibody-mediated neutralization.

### DISCUSSION

Since a significant fraction of the patient population with pre-existing antibodies to AAV capsids is currently ineligible to be enrolled in gene therapy trials with AAV vectors, and repeat dosing with AAV vectors is presently not possible, it is abundantly clear that novel strategies are needed to overcome this major barrier of vector neutralization. Indeed, efforts by a number of investigators are afoot to this end that include the following: (i) use of alternative AAV serotypes; (ii) transient depletion of B lymphocytes; (iii) structure-guided design of AAV vectors devoid of known antigenic epitopes; and (iv) site-directed mutagenesis of AAV capsids to develop AAV vectors capable of evading pre-existing antibodies.

In the present disclosure, AAV D-sequence-like sequences were shown to exist in the human genome, which, beyond computer-based homology searches, could also be isolated following PCR-based amplifications. These sequences were found in the following genes: INK4A/ARF, sorting nexin (SNX1), and lymphotoxin-α (LT-α). INK4A/ARF gene-encoded proteins are involved in pRB and p53 tumor suppressor pathways. Whether the well-known anti-tumor property of AAV involves the D-sequence, remains to be explored. Similarly, further studies are warranted to evaluate the role of SNX1 gene, known to encode a diverse group of cellular trafficking proteins and their propensity to form protein-protein complexes, in the life cycle of AAV. LT-α gene encodes a protein, also known as tumor necrosis factor-β (TNF-β), that possesses anti-proliferative activity.

One of these sequences was of significant interest since a cellular protein, which was termed as double-stranded D-sequence binding protein, could bind specifically to the X-box in the HLA-DRA promoter, and it was speculated that the dsDBP was a putative RFX transcription factor. Indeed, D-sequences from AAV1 and AAV2 viral genomes were recently shown to interact with RFX1 and RFX3 transcription factors. D-sequence in the AAV-ITR, which shares a partial sequence homology with the X-box in the HLA-DRA promoter, functionally competed for the binding of RFX transcription factor. This observation led to pursuit of a novel AAV vector in which one D-sequence in the ITR was replaced with an authentic X-box sequence in a scAAV2 vector. However, the extent of anti-AAV antibody production from both vectors following intravenous or intramuscular administration in a murine model was not significantly different. Similarly, intravitreal administration of X-box-containing scAAV2 vectors in one eye was insufficient to permit transduction by conventional AAV2 vectors in the contralateral or same eye in mice.

This apparent paradox between the in vitro and the in vivo data may be explained by the following: (i) AAV2 serotype vectors, known not to transduce murine antigen presenting cells were used; (ii) In contrast to the use of a large excess of D-sequence oligonucleotides in transfection experiments to achieve near total suppression of expression from the HLA-DRA promoter, AAV vectors containing only one X-box sequence were used for intravenous and intramuscular injections in vivo; (iii) scAAV2 and scAAV2-X-box vectors were used at only a 1:1 ratio for intravitreal injections; (iv) the number of resident antigen presenting cells in retina is generally low given the immune privileged status of this organ; and (v) D-sequence, and by inference, human X-box sequence, which interacts with human RFX1 and RFX3 transcription factors, was used in mouse models, whereas mouse X-box sequence is known to bind with mouse RFX5 transcription factor.

In summary, the administration of novel AAV vectors comprising X-box sequences of the HLA-DR promoter within the ITRs of the AAV genome may reduce the pre-existing AAV antibody titer, prevent immune responses, and allow for AAV administration in the setting of pre-existing immunity.

### Materials and Methods

### Cell Lines and Cultures

Human embryonic kidney, 293, and human cervical carcinoma, HeLa, cells were purchased from American Type Culture Collection (Manassas, Va.). Cells were maintained in complete Dulbecco Modified Eagle Medium (DMEM, Mediatech, Manassas, Va.) supplemented with 10% heat-inactivated fetal bovine serum (FBS, Sigma-Aldrich, St. Louis, Mo.) and 1% penicillin and streptomycin (P/S, Lonza, Walkersville, Md.). Cells were grown as adherent cultures in a humidified atmosphere at 37° C. in 5% CO₂, subcultured after treatment with trypsin-Versene mixture (Lonza, Walkersville, Md.) for 2 to 5 min at room temperature, washed, and resuspended in complete DMEM.

### Preparation of Whole Cell Extracts

Whole-cell extracts (WCEs) were prepared. Total protein concentration was determined with the Bio-Rad protein assay kit (Hercules, Calif.).

### Electrophoretic Mobility-Shift Assays

Electrophoretic mobility-shift (EMSA) assays were performed. Briefly, ³²P-labeled double-stranded oligonucleotides containing the AAV2 D-sequence (D), or the X-box sequence, were used as probes and incubated individually with whole cell extracts (WCE) prepared from 293 cells for 20 min at 25° C. and subjected to EMSA, in which the bound complexes were separated from the unbound probes on 6% polyacrylamide gels with 0.5×TBE buffer (pH 8.0) containing 89 mM Tris, 89 mM boric acid, 1 mM EDTA. Competition experiments using various unlabeled oligonucleotides were also performed. Following electrophoresis, gels were dried in vacuuo and autoradiographed with Kodak X-OMAT film.

### Molecular Cloning of Human D-Sequence-Like Sequences in the Human Genome

Human cellular D-sequence-like sequences were obtained by two consecutive rounds of PCR-amplification using five different commercially available human genomic libraries (Clontech, now Takara Bio, Mountain View, Calif.) with adapter primers (AP-1, 5′-GTAATACGACTCACTATAGGGC-3′ (SEQ ID NO: 14); AP-2, 5′-ACTATAGGGCACGCGTGGT-3 (SEQ ID NO: 15)) and the AAV D(−) sequence primer (5′-AGGAACCCCTAGTGATGGAG-3′ (SEQ ID NO: 16)). The PCR amplification products were subjected to nucleotide sequencing. Four sets of sequences were obtained and were found to be homologous to the INK4A/ARF promoter, sorting nexin 1 (SNX1), lymphotoxin-α (LT-α) regulatory region, and X-box in the human leukocyte antigen DRII type (HLA-DRA) promoter. These sequences were molecularly cloned in bacterial plasmids.

### Recombinant Plasmid Vectors

A recombinant plasmid containing the HLA-DRA promoter-driven firefly luciferase reporter gene (HLA-DRAp-Fluc) was constructed as follows. Briefly, a 467-bp DNA fragment (from −359 to +108) containing the HLA-DRA promoter was generated by PCR amplification using total human genomic DNA and the following primer-pair: 5′-ACGCAAACTCTCCAACTGTCATTGC-3′ (SEQ ID NO: 17), and 5′-TAGCACAGGGACTCCACTTATGGCC-3′ (SEQ ID NO: 18). The promoter sequence was cloned into a TA vector (Clontech). Following digestion with NdeI and SacI, a 378-bp DNA fragment (from −329 to +49) was isolated and ligated upstream of the FLuc reporter gene in the pGL3 cloning vector (Boehringer Mannheim, Indianapolis, Ind.). This resulted in a recombinant plasmid expressing the LacZ reporter gene under the control of the cytomegalovirus promoter (CMV-LacZ).

### DNA-Mediated Transfections and Quantitation of FLuc Expression In Vitro

293 or HeLa cells (1×10⁵ cells/well) in 12-well plates were transfected with pGL3 vector or HLA-DRAp-FLuc reporter plasmids, with or without synthetic oligonucleotides (10-200 ng) or AAV DNAs (4 μg) using the lipofection method. Transfected cells were washed with PBS, and cell lysates were prepared using 1× passive lysis buffer. Firefly luciferase (FLuc) activity was determined 48 hours post-transfections using an injector-equipped luminometer (BMG Labtech, FLUOstar Optima, Cary, N.C.), as recommended by the manufacturer.

### Recombinant AAV Vectors

Self-complementary AAV2 vectors expressing the enhanced green fluorescence protein (scAAV2-EGFP) or the mCherry (scAAV2-mCherry) reporter genes, and those containing the human X-box sequences (scAAV2-X-box-EGFP) were generated using the polyethyleneimine-mediated triple-plasmid transfection protocol. Briefly, 293 cells were co-transfected with three plasmids using polyethyleneimine (PEI, linear, MW 25000, Polyscinces, Inc., Warrington, Pa.), and medium was replaced 6 hours post-transfections. Cells were harvested 72 hours post-transfection, lysed by 3 rounds of freeze-thaw and digested with Benzonase (EMD Millipore, Darmstadt, Germany) at 37° C. for 1 hour. AAV vectors were purified by iodixanol (Sigma, St. Louis, Mo.) gradient ultracentrifugation followed by ion exchange column chromatography (HiTrap Q HP, 5 ml, GE Healthcare, Piscataway, N.J.), washed with phosphate-buffered saline (PBS) and concentrated by centrifugation using centrifugal spin concentrators (Apollo; 150 kDa cutoff, 20 ml capacity, Orbital Biosciences, Topsfield, Mass.). The physical genomic titers of highly purified scAAV vector stocks were determined by modified quantitative DNA slot blot analysis. Briefly, 10 l of vector stock was digested with Benzonase (EMD Millipore, Darmstadt, Germany) at 37° C. for 1 hour. An equal volume of 100 mM NaOH was added, followed by incubation at 65° C. for 30 min. A known quantity of plasmid DNA was denatured in the same manner for use as a reference standard for quantitation. Denatured DNA samples were loaded in two-fold serial dilutions onto Immobilon-NY+ membranes (Millipore, Bedford, Mass.). After UV cross-linking, the membranes were prehybridized for 1 hour at 42° C. in a hybridization solution containing 6×SSC, 100 μg/ml denatured herring sperm DNA, 0.5% sodium dodecyl sulfate (SDS), and 5×Denhardt's reagent. Subsequently, the membranes were hybridized with ³²P-labled DNA probes specific for EGFP or mCherry sequences in a hybridization solution at 42° C. for 18 to 20 hours. Membranes were washed twice with wash solution I (2×SSC, 0.1% SDS) at room temperature for 15 min, twice with wash solution II (0.5×SSC, 0.1% SDS) at 42° C. for 15 min, and then exposed to Amersham Typhoon RGB Biomolecular Imager (GE Healthcare, Chicago, Ill.) at room temperature. Autoradiography was performed as described above. Some vectors were also custom-packaged by SAB Tech, Inc. Philadelphia, Pa., and PackGene, Worcester, Mass. The vector titers and potencies were not significantly different from those produced by in-house methods.

### Intravascular and Intramuscular Injections in Mice

Six to 10-week-old C57BL6/J mice were purchased from Jackson Laboratory (Bar Harbor, Me.) and maintained by the Laboratory Animal Resource Center (LARC) at Indiana University School of Medicine, Indianapolis, Ind. Mice (n=4 per group) were injected either intravenously (IV) or intramuscularly (IM) with 1×10¹⁰ vgs of scAAV2-EGFP or scAAV2-X-box-EGFP vectors. Animals were kept in sterile cages until the end of the experiment. Mice were bled three weeks after vector administration and the level of anti-AAV2 IgG2c capsid antibodies were measured by ELISA. A multiple T test comparison between vectors did not show a statistical difference for IV (p=0.806) and IM (p=0.23) injected mice.

All animal experiments were approved by the Institutional Animal Care and Use Committee (IACUC) and performed according to the guidelines for animal care specified by the Laboratory Animal Resource Center (LARC) at Indiana University School of Medicine, Indianapolis, Ind.

### Ocular Injections

Intravitreal AAV vector injections were performed. C57BL6/J mice were intravitreally injected in their right eyes (RE) with 3×10¹¹ vg/mL of scAAV2-EGFP (n=8) or with scAAV2-X-box-EGFP (n=8) vectors (1 μl delivered). Extreme care was taken to ensure that the retina was not perforated during the procedure so as to avoid leakage of vector into the subretinal space. This was confirmed visually for each injection using a Nikon SMZ800 dissecting microscope fitted with an Olympus C-4040 camera and F1.8 super bright zoom lens. If a perforation was suspected, that eye was excluded from the study. Next, each cohort of 8 mice was sub-divided into two groups (n=4 each). In the first four mice, scAAV2-EGFP was injected intravitreally into the contralateral left eye (LE). In the second four mice, scAAV2-mCherry was injected into the same eye (RE).

### Fundoscopy

At 4 weeks post-injection, fundus images were captured using a Micron III camera (Phoenix Research Laboratories, Pleasanton, Calif.). Green and red fluorescent images were taken to visualize GFP and mCherry expression, respectively. Exposure settings remained constant between eyes. Four weeks later, fundus images were collected again, in the same manner.

### Statistical Analysis

All results are presented as mean±standard deviation (SD). Statistical analyses were performed using GraphPad Prism 7 software (GraphPad Software, Inc., San Diego, Calif.). Differences between groups were identified by grouped-unpaired two-tailed distribution of Student's T-test. A value of P<0.05 was considered statistically significant.

### OTHER EMBODIMENTS

All of the features disclosed in this specification may be combined in any combination. Each feature disclosed in this specification may be replaced by an alternative feature serving the same, equivalent, or similar purpose. Thus, unless expressly stated otherwise, each feature disclosed is only an example of a generic series of equivalent or similar features.

From the above description, one skilled in the art can easily ascertain the essential characteristics of the present disclosure, and without departing from the spirit and scope thereof, can make various changes and modifications of the disclosure to adapt it to various usages and conditions. Thus, other embodiments are also within the claims.

### EQUIVALENTS

While several inventive embodiments have been described and illustrated herein, those of ordinary skill in the art will readily envision a variety of other means and/or structures for performing the function and/or obtaining the results and/or one or more of the advantages described herein, and each of such variations and/or modifications is deemed to be within the scope of the inventive embodiments described herein. More generally, those skilled in the art will readily appreciate that all parameters, dimensions, materials, and configurations described herein are meant to be exemplary and that the actual parameters, dimensions, materials, and/or configurations will depend upon the specific application or applications for which the inventive teachings is/are used. Those skilled in the art will recognize, or be able to ascertain using no more than routine experimentation, many equivalents to the specific inventive embodiments described herein. It is, therefore, to be understood that the foregoing embodiments are presented by way of example only and that, within the scope of the appended claims and equivalents thereto, inventive embodiments may be practiced otherwise than as specifically described and claimed. Inventive embodiments of the present disclosure are directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the inventive scope of the present disclosure.

All definitions, as defined and used herein, should be understood to control over dictionary definitions, definitions in documents incorporated by reference, and/or ordinary meanings of the defined terms.

All references, patents and patent applications disclosed herein are incorporated by reference with respect to the subject matter for which each is cited, which in some cases may encompass the entirety of the document.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e. “one or the other but not both”) when preceded by terms of exclusivity, such as “either,” “one of,” “only one of,” or “exactly one of.” “Consisting essentially of,” when used in the claims, shall have its ordinary meaning as used in the field of patent law.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

It should also be understood that, unless clearly indicated to the contrary, in any methods claimed herein that include more than one step or act, the order of the steps or acts of the method is not necessarily limited to the order in which the steps or acts of the method are recited.

In the claims, as well as in the specification above, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of” and “consisting essentially of” shall be closed or semi-closed transitional phrases, respectively, as set forth in the United States Patent Office Manual of Patent Examining Procedures, Section 2111.03. It should be appreciated that embodiments described in this document using an open-ended transitional phrase (e.g., “comprising”) are also contemplated, in alternative embodiments, as “consisting of” and “consisting essentially of” the feature described by the open-ended transitional phrase. For example, if the disclosure describes “a composition comprising A and B”, the disclosure also contemplates the alternative embodiments “a composition consisting of A and B” and “a composition consisting essentially of A and B”.

What is claimed is:

1. A recombinant adeno-associated virus (rAAV) particle, comprising:
an rAAV nucleic acid comprising a 5′ inverted terminal repeat (ITR), an insert nucleic acid comprising a transgene, and a 3′ ITR, wherein the insert nucleic acid further comprises an HLA-DR promoter.

2. A recombinant adeno-associated virus (rAAV) particle, comprising:
an rAAV nucleic acid comprising a 5′ inverted terminal repeat (ITR), an insert nucleic acid comprising a transgene, a 3′ ITR, and one or more X-box sequences.

3. The rAAV particle of claim 2, wherein the one or more X-box sequences are located in the 5′ ITR and/or the 3′ ITR.

4. The rAAV particle of claim 2, wherein the one or more X-box sequences are located in the insert nucleic acid.

5. A recombinant adeno-associated virus (rAAV) particle, comprising:
an rAAV nucleic acid comprising a 5′ inverted terminal repeat (ITR), an insert nucleic acid comprising a transgene, a 3′ ITR, and one or more X2-box and/or Y-box sequences.

6. The rAAV particle of claim 5, wherein the one or more X2-box and/or Y-box sequences are in the 5′ ITR and the 3′ ITR.

7. The rAAV particle of claim 5, wherein the one or more X2-box and/or Y-box sequences are in the insert nucleic acid.

8. The rAAV particle of any one of claims 1-7, wherein the transgene is about 2- to 5-kb in length.

9. The rAAV particle of any one of claims 1-8, wherein the nucleic acid vector is a self-complementary AAV (scAAV) vector.

10. The rAAV particle of any one of claims 1-9, wherein the rAAV particle has a serotype that is selected from AAV2, AAV3, and AAV6, AAV3PM, AAV3QM, AAV6TM, AAV6QM, or a variant thereof.

11. The rAAV particle of claim 10, wherein the particle is an rAAV3 particle that comprises a modified capsid protein comprising a non-tyrosine residue at a position that corresponds to a surface-exposed tyrosine residue in a wild-type AAV3 capsid protein, a non-threonine residue at a position that corresponds to a surface-exposed threonine residue in the wild-type AAV3 capsid protein, a non-serine residue at a position that corresponds to a surface-exposed serine residue in the wild-type AAV3 capsid protein, or a combination thereof.

12. The rAAV particle of claim 10, wherein the particle is an rAAV2 particle that comprises a modified capsid protein comprising a non-tyrosine residue at a position that corresponds to a surface-exposed tyrosine residue in a wild-type AAV2 capsid protein, a non-threonine residue at a position that corresponds to a surface-exposed threonine residue in the wild-type AAV2 capsid protein, a non-serine residue at a position that corresponds to a surface-exposed serine residue in the wild-type AAV2 capsid protein, or a combination thereof.

13. The rAAV particle of claim 10, wherein the particle is an rAAV6 particle that comprises a modified capsid protein comprising a non-tyrosine residue at a position that corresponds to a surface-exposed tyrosine residue in a wild-type AAV6 capsid protein, a non-threonine residue at a position that corresponds to a surface-exposed threonine residue in the wild-type AAV6 capsid protein, a non-serine residue at a position that corresponds to a surface-exposed serine residue in the wild-type AAV6 capsid protein, a non-phenylalanine residue at position 129, or a combination thereof.

14. The rAAV6 particle of claim 13, further comprising a non-tyrosine residue, a non-threonine residue and a non-serine residue at each of Y705, Y731, T492 and S663 of a wild-type AAV6 capsid protein.

15. The rAAV6 particle of claim 13, further comprising Y445F, Y731F and F129L substitutions.

16. The rAAV particle of any one of claims 1-15, wherein the transgene encodes a therapeutic protein.

17. The rAAV particle of any one of claims 2-16, wherein the one or more X-box sequences differ by zero, one or two nucleotides relative to SEQ ID NO: 2.

18. The rAAV particle of any one of claims 5-16, wherein the one or more X2-box sequences differ by zero, one or two nucleotides relative to SEQ ID NO: 3.

19. The rAAV particle of any one of claims 5-16, wherein the one or more Y-box sequences differ by zero, one or two nucleotides relative to SEQ ID NO: 4.

20. A complex comprising the rAAV particle of any one of claims 1-19 and an RFX transcription factor, wherein the RFX is bound to the one or more X-box sequences, X2-box sequences and/or Y-box sequences.

21. A pharmaceutical composition comprising:
(a) the rAAV particle of any one of claims 1-19; and
(b) a pharmaceutically acceptable excipient.

22. The pharmaceutical composition of claim 21, wherein the composition is formulated for injection or topical application to a mammalian eye.

23. A method of transducing a cell, the method comprising administering an effective amount of the rAAV particle of any one of claims 1-19, or the pharmaceutical composition of claim 21 or 22, to the cell.

24. The method of claim 23, wherein the rAAV particle composition contains 3×10³-1×10⁴ vector genomes (vg)/mL of rAAV particles.

25. The method of claim 23 or 24, wherein the cell is a mammalian eye cell.

26. The method of claim 25, wherein the mammalian eye cell is a human cell.

27. The method of any one of claims 23-26, wherein the mammalian eye cell is selected from the group consisting of an ON retinal bipolar cell, an OFF retinal bipolar cell, a rod bipolar cell, and a cone bipolar cell.

28. The method of any one of claims 23-27, further comprising administering interferon-γ.


SEQUENCE LISTING (28 sequences)

SEQ ID NO: 1 - DNA, 378 bp, Homo sapiens
gggtacctcactaatgtgcttcaggtatatccctgtctagaagtcagattggggttaaagagtctgtccgtgattgactaacagtcttaaatacttgatttgttgttgttgttgtcctgtttgtttaagaactttacttctttatccaatgaacggagtatcttgtgtcctggaccctttgcaagaacccttcccctagcaacagatgcgtcatctcaaaatatttttctgattggccaaagagtaattgatttgcattttaatggtcagactctattacaccccacattctcttttcttttattcttgtctgttctgcctcactcccgagctctactgactcccaaaagagcgcccaagaagaaaccaccgcggtgg

SEQ ID NO: 2 - DNA, 20 bp, Homo sapiens
cccttcccctagcaacagat

SEQ ID NO: 3 - DNA, 9 bp, Homo sapiens
gatgcgtca

SEQ ID NO: 4 - DNA, 10 bp, Homo sapiens
ctgattggcc

SEQ ID NO: 5 - DNA, 145 bp, Artificial Sequence
  Synthetic Polynucleotide
ttggccactccctctctgcgcgctcgctcgctcactgaggccgggcgaccaaaggtcgcccgacgcccgggctttgcccgggcggcctcagtgagcgagcgagcgcgcagagagggagtggccaactccatcactaggggttcct

SEQ ID NO: 6 - DNA, 146 bp, Artificial Sequence
  Synthetic Polynucleotide
ttggccactccctctatgcgcactcgctcgctcggtggggcctggcgaccaaaggtcgccagacggacgtgctttgcacgtccggccccaccgagcgagcgagtgcgcatagagggagtggccaactccatcactagaggtatggc

SEQ ID NO: 7 - DNA, 142 bp, Artificial Sequence
  Synthetic Polynucleotide
ttgcccactccctctatgcgcgctcgctcgctcggtggggcctgcggaccaaaggtccgcagacggcagagctctgctctgccggccccaccgagcgagcgagcgcgcatagagggagtgggcaactccatcactaggggta

SEQ ID NO: 8 - DNA, 167 bp, Artificial Sequence
  Synthetic Nucleotide
ctctcccccctgtcgcgttcgctcgctcgctggctcgtttgggggggtggcagctcaaagagctgccagacgacggccctctggccgtcgcccccccaaacgagccagcgagcgagcgaacgcgacaggggggagagtgccacactctcaagcaagggggttttgta

SEQ ID NO: 9 - protein, 736 aa, Artificial Sequence
  Synthetic Polypeptide
MAADGYLPDWLEDNLSEGIREWWDLKPGAPKPKANQQKQDDGRGLVLPGYKYLGPFNGLDKGEPVNAADAAALEHDKAYDQQLKAGDNPYLRYNHADAEFQERLQEDTSFGGNLGRAVFQAKKRVLEPFGLVEEGAKTAPGKKRPVEQSPQEPDSSSGIGKTGQQPAKKRLNFGQTGDSESVPDPQPLGEPPATPAAVGPTTMASGGGAPMADNNEGADGVGNASGNWHCDSTWLGDRVITTSTRTWALPTYNNHLYKQISSASTGASNDNHYFGYSTPWGYFDFNRFHCHFSPRDWQRLINNNWGFRPKRLNFKLFNIQVKEVTTNDGVTTIANNLTSTVQVFSDSEYQLPYVLGSAHQGCLPPFPADVFMIPQYGYLTLNNGSQAVGRSSFYCLEYFPSQMLRTGNNFTFSYTFEDVPFHSSYAHSQSLDRLMNPLIDQYLYFLNRTQNQSGSAQNKDLLFSRGSPAGMSVQPKNWLPGPCYRQQRVSKTKTDNNNSNFTWTGASKYNLNGRESIINPGTAMASHKDDKDKFFPMSGVMIFGKESAGASNTALDNVMITDEEEIKATNPVATERFGTVAVNLQSSSTDPATGDVHVMGALPGMVWQDRDVYLQGPIWAKIPHTDGHFHPSPLMGGFGLKHPPPQILIKNTPVPANPPAEFSATKFASFITQYSTGQVSVEIEWELQKENSKRWNPEVQYTSNYAKSANVDFTVDNNGLYTEPRPIGTRYLTRPL

SEQ ID NO: 10 - DNA, 20 bp, Artificial Sequence
  Synthetic Polynucleotide
aggaacccctagtgatggag

SEQ ID NO: 11 - DNA, 20 bp, Artificial Sequence
  Synthetic Polynucleotide
ctccgttgctaggggaaggg

SEQ ID NO: 12 - DNA, 20 bp, Artificial Sequence
  Synthetic Polynucleotide
ctccatcactaggggttcct

SEQ ID NO: 13 - DNA, 20 bp, Artificial Sequence
  Synthetic Polynucleotide
atgcatcactaggggtccat

SEQ ID NO: 14 - DNA, 22 bp, Artificial Sequence
  Synthetic Polynucletoide
gtaatacgactcactatagggc

SEQ ID NO: 15 - DNA, 19 bp, Artificial Sequence
  Synthetic Polynucleotide
actatagggcacgcgtggt

SEQ ID NO: 16 - DNA, 20 bp, Artificial Sequence
  Synthetic Polynucleotide
aggaacccctagtgatggag

SEQ ID NO: 17 - DNA, 25 bp, Artificial Sequence
  Synthetic Polynucleotide
acgcaaactctccaactgtcattgc

SEQ ID NO: 18 - DNA, 25 bp, Artificial Sequence
  Synthetic Polynucleotide
tagcacagggactccacttatggcc

SEQ ID NO: 19 - DNA, 20 bp, Artificial Sequence
  Synthetic Polynucleotide
ctccatcactaggggttcct

SEQ ID NO: 20 - DNA, 20 bp, Artificial Sequence
  Synthetic Polynucleotide
gaggtagtgatccccaagga

SEQ ID NO: 21 - DNA, 20 bp, Artificial Sequence
  Synthetic Polynucleotide
tgacatcactaggggatact

SEQ ID NO: 22 - DNA, 20 bp, Artificial Sequence
  Synthetic Polynucleotide
actgtagtgatcccctatcc

SEQ ID NO: 23 - DNA, 20 bp, Artificial Sequence
  Synthetic Polynucleotide
ctccatcactaggggaatga

SEQ ID NO: 24 - DNA, 20 bp, Artificial Sequence
  Synthetic Polynucleotide
gaggtagtgatccccttact

SEQ ID NO: 25 - DNA, 20 bp, Artificial Sequence
  Synthetic Polynucleotide
atgcatcactaggggtccat

SEQ ID NO: 26 - DNA, 20 bp, Artificial Sequence
  Synthetic Polynucleotide
tacgtagtgatccccaggta

SEQ ID NO: 27 - DNA, 20 bp, Artificial Sequence
  Synthetic Polynucleotide
ctccgttgctaggggaaggg

SEQ ID NO: 28 - DNA, 20 bp, Artificial Sequence
  Synthetic Polynucleotide
gagacaacgatccccttccc